(19) United States
(12) Patent Application Publication
(10) Pub. No.: US 2022/0257704 A1
(43) Pub. Date: Aug. 18, 2022

(54) COMBINATION OF CHEMOTHERAPY WITH RECOMBINANT S. ROLFSII LECTIN

(71) Applicant: Unichem Laboratories Ltd, Mumbai, Maharashtra (IN)

(72) Inventors: Dhananjay SATHE, Maharashtra (IN); Sudeep KUMAR, Gujarat (IN); Sachin Prabhakar BACHATE, Maharashtra (IN); Mohanish Pradeep KANKONKAR, Goa (IN)

(73) Assignee: Unichem Laboratories Ltd, Mumbai, Maharashtra (IN)

(21) Appl. No.: 17/294,570

(22) PCT Filed: Nov. 18, 2019
(86) PCT No.: PCT/IB2019/059873

§ 371 Date: May 17, 2021

(30) Foreign Application Priority Data
Nov. 19, 2018 (IN) 201821043455
Dec. 18, 2018 (IN) 201821022667

(51) Int. Cl. A61K 38/16; A61K 33/243; A61P 35/00; A61K 31/435; A61K 31/495

(52) U.S. Cl. CPC A61K 38/168; A61K 33/243; A61K 2121/00; A61K 31/435; A61K 31/495; A61P 35/00

(57) ABSTRACT

The present invention relates to the combination chemotherapy with recombinant lectin protein. The invention specifically relates to the cytotoxic effect of recombinant lectin protein having amino acid sequence of SEQ ID NO: 1 in combination with other therapeutic agents, wherein the other therapeutic agents are anti-cancer agents. The combinations are highly synergistic and efficacious against several cancers.

### CROSS REFERENCE TO RELATED APPLICATIONS

The application claims the benefit of Indian Provisional Application No. 201821022667 filed on Dec. 18, 2018 and 201821043455 filed on Nov. 19, 2018, the entire contents of which are incorporated herein by reference.

### FIELD OF INVENTION

The present invention relates to biopharmaceutical combinations of recombinant lectin protein with other therapeutic agents and their antiproliferative effects towards controlling uncontrolled proliferation of cells in cancers.

### BACKGROUND OF INVENTION

A cancer starts with a primary tumor, which is swelling or morbid enlargement that results from an overabundance of cell growth and division. Tumors may be benign, pre-malignant or malignant, or can signify a lesion with no cancerous potential. A benign tumor is a non-cancerous tumor and a malignant tumor is a cancerous tumor. Cancer is when abnormal cells divide in an uncontrolled way. Cancer can sometimes spread to other parts of the body—this is called a secondary tumor or a metastasis.

Cancer treatment therapies include surgical removal of the cancerous tissues, chemotherapy, polychemotherapy, radiotherapy, immunotherapy, etc. These therapies are often invasive techniques which also affect the normal healthy cells along with the cancer cells. Also these therapies are used in combination in order to achieve synergistic effect resulting in enhancing the therapeutic potential of constituent drugs, decreasing the dose, reducing the toxicity of the drugs and delaying induction of drug resistance.

Chemotherapies for cancer treatment are decided based on the mode of cancer cell proliferation. Different properties of the chemotherapeutic agents such as anti-metabolic agents, which interfere with the production or use of metabolites required by cancers; anti-microtubule agents, which interfere with micro-tubule formation, necessary for mitosis; alkylating agents, which are able to interfere with DNA alkylation by blocking DNA replication; platinum based agents which are able to cross-link DNA; antitumor antibiotic agents, and topoisomerase inhibitors, which inhibit the topoisomerase enzyme necessary for DNA replication.

Oncofetal Thomsen-Friedenreich antigen (Galβ1-3GalNAc-α-O-Ser/Thr, T or TF), which is expressed in more than 90% of cancers including oral cancer, colon cancer, ovarian cancer as well as bladder cancer and expression of TF is correlated with tumor progression and metastasis. Applicant's previous patent application 350/MUM/2009 discloses a recombinant lectin protein isolated from sclerotial bodies of fungus Sclerotium rolfsii having high binding specificity toward the oncofetal TF carbohydrate antigen.

A combination therapy for cancer treatment is an effective treatment due to undesirable cytotoxicity of healthy cells. The complexity of the disease, its tendency to spread beyond its original site and become resistant to certain drugs and its genetic diversity underscores the need for a variety of approaches to attack it. Combination therapy not only increases the chances of a cure or long-term remission, but also reduces damage to vital organs and tissues more than a single approach. One type of therapy can sometimes make a tumor more vulnerable to a second type. Certain chemotherapy drugs, for example, can increase tumors' vulnerability to radiation therapy. In other cases, drugs may have a synergistic effect when administered together and enhance each other's potency, so the combined effectiveness is greater than the individual impact. Using them in combination has a multiplier effect: their benefits are magnified many times over. U.S. Pat. No. 5,053,386 (1991) refers to the composition and methods of treatment comprising lectins, Abrin and Abrus Agglutinin for the suppression of post-surgical malignant tumor metastasis. It also discloses the use of a protein in combination with either or both radiation treatment and/or chemotherapy.

Molecular and cellular biochemistry (Vol—394 (1-2); Page—225-235; Year—2014) discloses the effects of lectin from Korean mistletoe (Viscum album var. coloratum agglutinin, VCA) and doxorubicin (DOX) in MCF-7 (estrogen receptor-positive) and MDA-MB231 (estrogen receptor-negative) human breast cancer cells, in combination and alone. Combination of VCA and DOX showed, a strong synergistic effect in cell growth inhibition, compared to VCA or DOX treatment alone.

Oncotarget (Vol: 8 (26), Page: 42466-42477, Year: 2017) discloses combination treatment with pemetrexed and Sialic acid-binding lectin isolated from Ranacatesbeiana oocytes (cSBL) resulting in greater dose-dependent cytotoxicity than combination of pemetrexed and cisplatin, the standard of care in mesothelioma.

However, the difficulty is not all these combination therapies result in beneficial effects. Hence presently research is focused on developing new and useful anti-proliferative combination partners.

To fight a life-threatening disease like cancer, it is the need of an hour to develop best method for its prevention and treatment. Combination therapy seems to be one of such options.

### OBJECT OF THE INVENTION

The main object of the present invention is to provide an alternative method to treat or prevent growth of cancer cells. More specifically the objective is to develop an affordable and efficient method to treat or prevent growth of cancer cells.

Another object of the present invention is to provide an effective therapy for prevention or treatment of cancer. Precisely the objective of the present invention is to provide a combination therapy for the prevention or treatment of cancer, wherein the combination is a synergistic combination comprising a recombinant lectin protein.

Yet another object of the present invention is to provide a therapeutically effective combination for the prevention and/or treatment of cancer.

Another object of the invention is to provide an effective concentrations of the therapeutically effective synergistic combination for the prevention and/or treatment of cancer.

Yet another object of the invention is to provide a pharmaceutically acceptable formulation of effective concentrations of the therapeutically effective combination for the prevention and/or treatment of cancer.

Yet another object of the invention is to provide combinations of recombinant lectin protein having amino acid sequence of SEQ ID NO: 1 useful in the prevention, treatment or inhibition of proliferation of cancer cells.

### SUMMARY OF INVENTION

The main aspect of the present invention is the therapeutically effective combination comprising recombinant lectin protein and one or more other therapeutic agent.

Preferably, the combination is synergistic.

Preferably, the concentration of recombinant lectin protein is in the range from 0.5 μg/mL to 100 μg/mL.

Preferably, the therapeutically effective combination is used for prevention or treatment of cancer in a subject.

According to a second aspect of the present invention, there is provided a recombinant lectin protein for the treatment or prevention of cancer in a subject wherein the recombinant lectin protein is administered in combination with one or more other therapeutic agent, and wherein the other therapeutic agent is administered simultaneously, separately or sequentially.

According to a third aspect of the present invention, there is provided a method of treatment or prevention of cancer or inhibition of growth of tumor cells in a subject comprising administering to the subject effective amount of recombinant lectin protein in combination with one or more other therapeutic agent, wherein the other therapeutic agent is administered simultaneously, separately or sequentially.

According to a fourth aspect of the present invention, there is provided a combination therapy for prevention, proliferation, treatment or to cure cancer or tumor in a subject comprising administration of recombinant lectin protein either in combination with one or more other therapeutic agent, and wherein the other therapeutic agent is administered simultaneously, separately, or sequentially.

Advantageously, the other therapeutic agent is an anticancer agent.

Preferably, the anti-cancer agent is an antitumor antibiotic.

Preferably, the anti-cancer agent is an antimetabolite, an alkylating antineoplastic agent, an anti-microtubule agent and/or a topoisomerase I inhibitor.

More preferably, the anti-cancer agent is an oxazaphosphorine, a nitrogen mustard, alkylating anti-neoplastic agent, a topoisomerase I inhibitor, a topoisomerase II inhibitor, a vinca alkaloid, a taxane, an antifolate, or a pyrimidine antagonist.

More preferably, the antimetabolite is selected from 5-Flurouracil (5-FU), Gemcitabine, Methotrexate, Pemetrexed or Capecitabine.

More preferably, the alkylating anti-neoplastic agent is a platinum based anti-neoplastic agent selected from cisplatin or carboplatin.

More preferably, the anti-microtubule agent is selected from Paclitaxel, Docetaxel, Abraxane or Taxotere.

More preferably, the topoisomerase I inhibitor is selected from Irinotecan or Topotecan.

Advantageously, the anticancer agent is cisplatin and the combination is used for the treatment or prevention of oral cancer, ovary cancer or bladder cancer in a subject.

Preferably wherein the cancer is oral cancer and the concentration of the recombinant lectin is 10 to 90 μg/mL and the concentration of cisplatin is 0.1 to 1.5 μM, or wherein the concentration of the recombinant lectin is 5 μg/mL and the concentration of cisplatin is 0.5 to 1.5 μM.

Preferably wherein the cancer is ovary cancer and the concentration of the recombinant lectin is 1 to 2.5 μg/mL and the concentration of cisplatin is 0.25 to 5 μM, or wherein the concentration of the recombinant lectin is 5 μg/mL and the concentration of cisplatin is 0.1 to 5 μM, or wherein the concentration of the recombinant lectin is 10 to 20 μg/mL and the concentration of cisplatin is 0.01 to 5 μM.

Preferably wherein the cancer is bladder cancer and the concentration of the recombinant lectin is 5 to 80 μg/mL and the concentration of cisplatin is 1 to 500 μM

Advantageously, the anticancer agent is 5-FU and the combination is used for the treatment or prevention of oral, pancreatic or colon cancer.

Preferably wherein the cancer is oral cancer and the concentration of the recombinant lectin is 5 to 90 μg/mL and the concentration of 5-FU is 0.01 to 5 μM.

Preferably wherein the cancer is pancreatic cancer and the concentration of the recombinant lectin is 10 to 80 μg/mL and the concentration of 5-FU is 1 to 250 μM, or wherein the concentration of the recombinant lectin is 5 μg/mL and the concentration of 5-FU is 1 to 50 or 250 μM.

Preferably wherein the cancer is colon cancer and the concentration of the recombinant lectin is 5 to 80 μg/mL and the concentration of 5-FU is 10 to 200 μM, or wherein the concentration of the recombinant lectin is 1 μg/mL and the concentration of 5-FU is 50 to 200 μM;

Advantageously, the anticancer agent is Irinotecan and the combination is used for the treatment or prevention of colon cancer.

Preferably wherein the concentration of the recombinant lectin is 40 to 80 μg/mL and the concentration of Irinotecan is 1 to 50 μM, or wherein the concentration of the recombinant lectin is 20 μg/mL and the concentration of Irinotecan is 1 or 10 to 50 μM.

Advantageously, the anticancer agent is Paclitaxel for the treatment or prevention of ovary or breast cancer.

Preferably wherein the cancer is ovary cancer and the concentration of the recombinant lectin is 1 to 20 μg/mL and the concentration of Paclitaxel is 0.25 to 5 nM.

Preferably wherein the cancer is breast cancer and the concentration of the recombinant lectin is 20 to 80 μg/mL and the concentration of Paclitaxel is 0.1 to 10 nM; or wherein the concentration of the recombinant lectin is 10 μg/mL and the concentration of Paclitaxel is 1 to 10 nM.

Advantageously, the anticancer agent is Gemcitabine and the combination is used for the treatment or prevention of bladder or pancreatic cancer.

Preferably wherein the cancer is bladder cancer and the concentration of the recombinant lectin is 2.5 to 80 μg/mL and the concentration of Gemcitabine is 1 to 300 μM.

Preferably wherein the cancer is pancreatic cancer and the concentration of recombinant lectin is 5 to 20 μg/mL and the concentration of Gemcitabine is 5 to 25 μM; or wherein the concentration of the recombinant lectin is 40 to 80 μg/mL and the concentration of Gemcitabine is 1 to 25 μM.

Advantageously, the anticancer agent is carboplatin and the combination is used for the treatment or prevention of breast cancer;

Preferably wherein the concentration of the recombinant lectin is 5 to 80 μg/mL and the concentration of Carboplatin is 50 to 1000 μM; or wherein the concentration of the recombinant lectin is 2.5 μg/mL and the concentration of Carboplatin is 100 to 1000 μM.

Advantageously, the recombinant lectin protein is a protein having amino acid sequence of SEQ ID NO: 1, 2 or 3 or having at least 60%, 70%, 80%, 90%, 95%, 95%, 96%, 97%, 98%, or 99% homology to SEQ ID NO: 1, 2 or 3.

Preferably, the concentration of the recombinant lectin protein is in the range from 0.5 μg/mL to 100 μg/mL. More preferably, the concentration of the recombinant lectin protein is in the range from 1 μg/mL to 90 μg/m L.

Preferably, the concentration of the other therapeutic agent is in the range from 0.001 nM to 1000 μM.

Preferably, the concentration of Cisplatin is in the range of 0.01 μM to 500 μM.

Preferably, the concentration of 5-Flurouracil (5-FU) is in the range of 0.01 μM to 250 μM.

Preferably, the concentration of Irinotecan is in the range of 0.1 μM to 50 μM.

Preferably, the concentration of Paclitaxel is in the range of 0.001 nM to 10 nM.

Preferably, the concentration of Gemcitabine is in the range of 0.01 μM to 300 μM.

Preferably, the concentration of Carboplatin is in the range of 10 μM to 1000 μM.

Advantageously, the combination comprises a dosage of the recombinant lectin and a dosage of the other therapeutic agent suitable for achieving a therapeutic effect.

Preferably, the dosage of the recombinant lectin is suitable for achieving a concentration of the recombinant lectin in vivo of 0.5 μg/mL to 100 μg/mL.

Preferably, the dosage of the other therapeutic agent is suitable for achieving a concentration of the other therapeutic agent in vivo of 0.001 nM to 1000 μM.

Preferably, the dosage of Cisplatin is suitable for achieving a concentration of Cisplatin in vivo of 0.01 μM to 500 μM.

Preferably, the dosage of 5-Flurouracil (5-FU) is suitable for achieving a concentration of 5-Flurouracil (5-FU)in vivo of 0.01 μM to 250 μM.

Preferably, the dosage of Irinotecan is suitable for achieving a concentration of Irinotecan in vivo of 0.1 μM to 50 μM.

Preferably, the dosage of Paclitaxel is suitable for achieving a concentration of Paclitaxel in vivo of 0.001 nM to 10 nM.

Preferably, the dosage of Gemcitabine is suitable for achieving a concentration of Gemcitabine in vivo of 0.01 μM to 300 μM.

Advantageously, the combination, of the recombinant lectin protein and the other therapeutic agent, is a composition. Preferably, the composition further comprises one or more pharmaceutically acceptable excipients.

According to a fifth aspect of the invention, there is provided a recombinant lectin protein in combination with one or more other therapeutic agent for the treatment or prevention of cancer wherein the other therapeutic agent is selected from one or more of 5-Flurouracil (5-FU), Gemcitabine, cisplatin, Paclitaxel, carboplatin or Irinotecan.

Preferably, the recombinant lectin is for the treatment of oral cancer, ovary cancer or bladder cancer, wherein recombinant lectin protein is administered in combination with Cisplatin, and wherein Cisplatin is administered simultaneously, separately or sequentially.

Preferably, the recombinant lectin is for the treatment of oral, pancreatic or colon cancer, wherein recombinant lectin protein is administered in combination with 5-FU, and wherein 5-FU is administered simultaneously, separately or sequentially.

Preferably, the recombinant lectin is for the treatment of colon cancer, wherein recombinant lectin protein is administered or in combination with Irinotecan, and wherein Irinotecan is administered simultaneously, separately or sequentially.

Preferably, the recombinant lectin is for the treatment of ovary cancer or breast cancer, wherein recombinant lectin protein is administered or in combination with Paclitaxel, and wherein Paclitaxel is administered simultaneously, separately or sequentially.

Preferably, the recombinant lectin is for the treatment of bladder cancer or pancreatic cancer, wherein recombinant lectin protein is administered or in combination with Gemcitabine, and wherein Gemcitabine is administered simultaneously, separately or sequentially.

Preferably, the recombinant lectin is for the treatment of breast cancer, wherein recombinant lectin protein is administered or in combination with carboplatin, and wherein carboplatin is administered simultaneously, separately or sequentially.

According to a sixth aspect of the invention, there is provided a therapeutic agent for the treatment or prevention of cancer, in combination with a recombinant lectin protein, wherein the therapeutic agent is selected from one or more of 5-Flurouracil (5-FU), Gemcitabine, Cisplatin, Paclitaxel, Carboplatin or Irinotecan.

Preferably, the therapeutic agent is for the treatment of oral cancer, ovary cancer or bladder cancer, wherein the therapeutic agent is Cisplatin, and wherein the Cisplatin is administered in combination with the recombinant lectin protein, and wherein the recombinant lectin protein is administered simultaneously, separately or sequentially.

Preferably, the therapeutic agent is for the treatment of oral, pancreatic or colon cancer, wherein the therapeutic agent is 5-FU, and wherein the 5-FU is administered in combination with the recombinant lectin protein, and wherein the recombinant lectin protein is administered simultaneously, separately or sequentially.

Preferably, the therapeutic agent is for the treatment of colon cancer, wherein the therapeutic agent is Irinotecan, and wherein the Irinotecan is administered in combination with the recombinant lectin protein, and wherein the recombinant lectin protein is administered simultaneously, separately or sequentially.

Preferably, the therapeutic agent is for the treatment of ovary cancer or breast cancer, wherein the therapeutic agent is Paclitaxel, and wherein the Paclitaxel is administered in combination with the recombinant lectin protein, and wherein the recombinant lectin protein is administered simultaneously, separately or sequentially.

Preferably, the therapeutic agent is for the treatment of bladder cancer or pancreatic cancer, wherein the therapeutic agent is Gemcitabine, and wherein the Gemcitabine is administered in combination with the recombinant lectin protein, and wherein the recombinant lectin protein is administered simultaneously, separately or sequentially.

Preferably, the therapeutic agent is for the treatment of breast cancer, wherein the therapeutic agent is Carboplatin, and wherein the Carboplatin is administered in combination with the recombinant lectin protein, and wherein the recombinant lectin protein is administered simultaneously, separately or sequentially.

### DESCRIPTION OF INVENTION

### Definitions

The term “protein” as used herein refers to a polymer of amino acid residues.

The term “amino acid” as used herein refers to naturally occurring and synthetic amino acids, as well as amino acid analogues and amino acid mimetics that have a function that is similar to the naturally occurring amino acids. Naturally occurring amino acids are those encoded by the genetic code and include the proteinogenic amino acids. Naturally occurring amino acids also include those modified after translation in cells. Synthetic amino acids include non-canonical amino acids such as selenocysteine and pyrrolysine. Typically synthetic amino acids are not proteinogenic amino acids.

The terms “Cancer” and “Tumor”, are interchangeably used in the present invention, are having same meaning as understood by the person skilled in the art. They form when the normal cells grow out of control and crowd out healthy body cells. Formation of such an enlarged outgrowth affects the normal functioning of the tissue/organ/organism. Cancer can start any place in the body and can also spread to other parts of the body. When cancer cells spread within the body, it is called metastasis.

The term “recombinant” means a nucleic acid or a polypeptide has been artificially or synthetically (i.e., non-naturally) altered by human intervention. The alteration can be performed on the material within, or removed from, its natural environment or state. For example, a “recombinant nucleic acid” is one that is made by recombining nucleic acids, e.g., during cloning, DNA shuffling or other well-known molecular biological procedures. A “recombinant DNA molecule” is comprised of segments of DNA joined together by means of such molecular biological techniques. The term “recombinant protein” or “recombinant polypeptide” as used herein refers to a protein molecule which is expressed using a recombinant DNA molecule.

The term “lectin” as used herein refers to a carbohydrate-binding protein. More specifically the recombinant lectin protein of the present invention is the lectin derived from Sclerotium rolfsii lectin (SRL). Sclerotium rolfsii lectin (SRL) is a lectin that has been isolated from the sclerotial bodies of the soil-borne phytopathogenic fungus S. rolfsii.

The terms “homology” or “homologous” as used herein refer to two or more referenced entities that share at least partial identity over a given region or portion. Areas, regions or domains of homology or identity refer to a portion of two or more referenced entities that are similar or are the same. Thus, where two sequences are identical over one or more sequence regions they share identity in these regions. Substantial homology refers to a molecule that is structurally or functionally conserved such that it has, or is predicted to have, at least a similar structure or function (e.g., a biological function or activity) to a reference molecule. Or it may means that the molecule has a relevant/corresponding region or portion which it shares homology with that of the reference molecule.

In one embodiment, the percentage “homology” between two sequences is determined using the BLASTP algorithm with default parameters (Altschul et al. Nucleic Acids Res. 1997 Sep. 1; 25(17):3389-402). In particular, the BLAST algorithm can be accessed on the internet using the URL: https://blast.ncbi.nlm.nih.gov/Blast.cgi. In an alternative embodiment, for global sequence alignments, percentage homology between two sequences is determined using the EMBOSS Needle algorithm using default parameters. In particular, the EMBOSS Needle algorithm can be accessed on the internet using the URL: https://www.ebi.ac.uk/Tools/psa/emboss_needle/.

Unless otherwise indicated, the term “homology” is used interchangeably with the term “sequence identity” in the present specification.

The term “recombinant lectin protein” is intended here to cover any pharmaceutically acceptable salt, solvate, hydrate, prodrug, or any other compound which, upon administration to the patient is capable of providing (directly or indirectly) the compound as described herein. The preparation of salts, solvates, hydrates, and prodrugs can be carried out by methods known in the art.

As used herein, the terms “therapies” and “therapy” can refer to any method, composition, and/or active ingredient that can be used in the treatment, prevention and/or management of the disease or one or more symptoms thereof.

The term “combination therapy” as used throughout the specification, is meant to encompass the administration to a patient suffering from cancer of the referred therapeutic agents in the same or separate pharmaceutical formulations, and at the same time or at different times. If the therapeutic agents are administered at different times they should be administered sufficiently close in time to provide for the potentiating or synergistic response to occur.

The term “chemotherapy” refers to the use of drugs to treat cancer. As used herein, a “chemotherapeutic agent” is a chemical compound useful in the treatment of cancer.

The therapeutic agent that may be used in combination with recombinant lectin protein is preferably a chemotherapeutic agent. There are no limitations on the chemotherapeutic agent (the ‘term’ chemotherapeutic agent has the same meaning as that known to a person skilled in the art) that can be used in this invention. Non limiting examples of such chemotherapeutic agents are alkylating agents such as, e.g., mitomycin C, cyclophosphamide, busulfan, ifosfamide, isosfamide, melphalan, hexamethylmelamine, thiotepa, chlorambucil, mechlorethamine or dacarbazine; antimetabolites such as, e.g., gemcitabine, capecitabine, 5-fluorouracil, cytarabine, 2-fluorodeoxy cytidine, methotrexate, idatrexate, tomudex or trimetrexate; topoisomerase II inhibitors such as, e.g., doxorubicin, epirubicin, etoposide, teniposide or mitoxantrone; topoisomerase I inhibitors such as, e.g., irinotecan (CPT-11), 7-ethyl-10-hydroxy-camptothecin (SN-38) or topotecan; antimitotic drugs such as, e.g., paclitaxel, docetaxel, vinblastine, vincristine or vinorelbine; and platinum derivatives such as, e.g., cisplatin, oxaliplatin, spiroplatinum or carboplatinum.

Chemotherapeutic agents can be broadly classified as described above. They can also be further classified into subclasses as described in the table below.

| Main class | Subclass | Chemotherapeutic agents |
|---|---|---|
| Alkylating agents | Oxazaphosphorines | cyclophosphamide, ifosfamide, |
| | | isosfamide, |
| | Nitrogen mustards | melphalan, chlorambucil, |
| | | busulfan, mechlorethamine |
| | Platinum-based | Cisplatin, Carboplatin, |
| | agents | Oxaliplatin, spiroplatinum |
| | Others | thiotepa, dacarbazine; |
| | | mitomycin C, |
| | | hexamethylmelamine |
| Topoisomerase | Topoisomerase I | irinotecan (CPT-11), 7-ethyl- |
| inhibitors | | 10-hydroxy-camptothecin |
| | | (SN-38) or topotecan |
| | Topoisomerase II | doxorubicin, epirubicin, |
| | | etoposide, teniposide |
| | | or mitoxantrone; |
| Mitotic inhibitors | Vinca alkaloids | vinblastine, vincristine |
| | | or vinorelbine; |
| | Taxanes | paclitaxel, docetaxel, |
| Antimetabolites | Antifolates | methotrexate, or trimetrexate; |
| | | tomudex |
| | Pyrimidine | gemcitabine, capecitabine, |
| | antagonists | 5-fluorouracil, cytarabine, |
| | | 2-fluorodeoxy cytidine, |
| | Others | idatrexate, |

The term “radiation therapy” or “radiotherapy” is the treatment of cancer and other diseases with ionizing radiation. Ionizing radiation deposits energy that injures or destroys cells in the area being treated by damaging their genetic material, making it impossible for these cells to continue to grow. Although radiation damages both cancer cells and normal cells, the latter are able to repair themselves and function properly. Radiation therapy used according to the present invention may include, but is not limited to, the use of gamma-rays, X-rays, and/or the directed delivery of radioisotopes to tumor cells. Other forms of DNA damaging factors are also contemplated such as microwaves and UV-irradiation. It is further contemplated that radiotherapy may include the use of radiolabeled antibodies to deliver doses of radiation directly to the cancer site (radioimmunotherapy) and/or include the use of a radiosensitizer.

The term “immunotherapy” rely on the use of immune effector cells and molecules to target and destroy cancer cells. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. The combination of therapeutic modalities, i.e., direct cytotoxic activity and inhibition or reduction of ErbB2 would provide therapeutic benefit in the treatment of ErbB2 overexpressing cancers.

As used herein, “Pharmaceutically-acceptable salt” refers to salts which retain the biological effectiveness and properties of compounds which are not biologically or otherwise undesirable. Pharmaceutically-acceptable salts refer to pharmaceutically-acceptable salts of the compounds, which salts are derived from a variety of organic and inorganic counter ions well known in the art.

The term “pharmaceutical combination” refers to any combination of a first and a second pharmaceutical ingredient, whether mixed into a single composition or maintained separately. The term “pharmaceutical composition” or “pharmaceutically acceptable composition” or “pharmaceutically acceptable formulation” refers to a mixture of a compound disclosed herein with pharmaceutical excipients, such as diluents or carriers (see, for example, Remington: The Science and Practice of Pharmacy 22nd ed., Pharmaceutical Press (Sep. 15, 2012) and handbook of Pharmaceutical Excipients, 6th edition, Raymond Rowe, Pharmaceutical Press (2009)). The pharmaceutical composition facilitates administration of the compound to an organism. Pharmaceutical compositions will generally be tailored to the specific intended route of administration.

The terms “effective” or “therapeutically effective” refer to an effect sufficient to elicit the desired biological response. As will be appreciated by those of ordinary skill in this art, the effect of an inventive combination may vary depending on such factors as the desired biological endpoint, the pharmacokinetics of the agents being delivered, the disease being treated, the mode of administration, and the patient. Treatment is generally “effective” if one or more symptoms or clinical markers are reduced. Alternatively, treatment is “effective” if the progression of a disease, disorder or medical condition is reduced or halted.

Combination Index (CI) values were based on Loewe's additivity model were determined to assess the nature of drug-drug interactions that can be additive (CI=1), antagonistic (CI>1), or synergistic (CI<1) for various drug-drug concentrations and effect levels (Fa, fraction affected; inhibition of cancer cell proliferation). CI values were calculated based on linear regression trendlines using the CompuSyn software (ComboSyn Inc., Paramus, N.J.), following the method by Chou et al., whereby the hyperbolic and sigmoidal dose-effect curves are transformed into a linear form (Chou T C (2010) Drug combination studies and their synergy quantification using the Chou-Talalay method. Cancer Res 70: 440-6, instructions also available at ComboSyn, Inc., www.combosyn.com).

A synergistic effect permits the effective treatment of a disease using lower amounts (doses) of individual therapy. The lower doses result in lower toxicity without reduced efficacy. In addition, a synergistic effect can result in improved efficacy. Finally, synergy may result in an improved avoidance or reduction of disease as compared to any single therapy. The term “additive” refers to sum of any two or more therapeutic agents in combination. As used herein, the term “antagonistic” refers to block (e.g., reduces or prevents) a biological activity. The term “inhibit” or “inhibition” means to reduce by a measurable amount.

As used herein, the term “anti-cancer agent” refers to a substance or treatment (e.g., radiation therapy) that inhibits the function of cancer cells, inhibits their formation, and/or causes their destruction in vitro or in vivo.

The term “antitumor antibiotic” refers to a chemical substance that has an antitumor effect. In some embodiments, “antitumor antibiotic” referred to herein is not an anti-bacterial antibiotic used to treat infections. It is a type of anticancer drug that blocks cell growth by interfering with DNA.

The term “Cytotoxic” is used in context with the effect of therapeutic agents of the present invention on the cancer cell lines and means ‘toxic to cells’. The therapeutic agents that are effective in destroying the cancer cells are called cytotoxic or described as having cytotoxicity.

The term “IC50” or IC₅₀ refers to an inhibitory dose which causes 50% inhibition of a given quantifiably measureable parameter. This quantitative measure indicates how much of a particular drug or other substance (inhibitor) is needed to inhibit a given biological, biochemical or chemical parameter(or component of a parameter, i.e. an enzyme, cell, cell receptor or microorganism) by half.

The term “synergistic” refers to a combination of two or more therapeutic agents, which is more effective than the additive effects of any two or more single agents. Synergy of pharmacological agents can be determined quantitatively by calculating Combination Index (CI) values.

CI values can be calculated based on the measured IC50 values for two drugs A and B, using the formula as given in below.

$$CI = \frac{{DA}_{( 50 )}}{{ICA}_{( 50 )}} + \frac{{DB}_{( 50 )}}{{ICB}_{( 50 )}} + \frac{{DA}_{( 50 )} \times {DB}_{( 50 )}}{{ICA}_{( 50 )} \times {ICB}_{( 50 )}}$$

Where,

DA₍₅₀₎=Concentration of drug A in combination with B to produce 50% cytotoxicity

ICA₍₅₀₎=Concentration of drug A alone to produce 50% cytotoxicity

DB₍₅₀₎=Concentration of drug B in combination with A to produce 50% cytotoxicity

ICB₍₅₀₎=Concentration of drug B alone to produce 50% cytotoxicity

Wherein drug A is a lectin of the invention (e.g. a protein of SEQ ID NO: 1) and drug B is the other therapeutic agent that was tested.

Synergism, additive or antagonism is determined based on the following criteria:
CI>1—antagonism is indicated
CI=1—additive effected is indicated
CI<1—synergism is indicated

As such, if the CI value is calculated to be less than one, this indicates that drugs A and B have a synergistic effect.

As used herein, a “subject” refers to an animal that is the object of treatment, observation or experiment. “Animal” includes cold- and warm-blooded vertebrates and invertebrates such as fish, shellfish, reptiles and, in particular, mammals. “Mammal” includes, without limitation, mice, rats, rabbits, guinea pigs, dogs, cats, sheep, goats, cows, horses, primates, such as monkeys, chimpanzees, and apes, and, in particular, humans.

In addition, any drug referred to herein may be in amorphous form or crystalline form either as free compound or as solvates (e.g. hydrates) and it is intended that both forms are within the scope of the present invention. Methods of solvation are generally known within the art.

### Brief Description of the Accompanying Sequences

SEQ ID NO: 1: represents a variant of the S. rolfsii lectin amino acid sequence (reported as Rec-2 in WO 2010/095143).

SEQ ID NO: 2: represents a variant of the S. rolfsii lectin amino acid sequence (reported as Rec-3 in WO 2010/095143).

SEQ ID NO: 3: represents a variant of the S. rolfsii lectin amino acid sequence (reported in WO 2014/203261).

Recombinant lectin protein having Amino acid
sequence of SEQ ID NO: 1
TYKIT VRVYQ TNPDA FFHPV EKTVW KYANG GTWTI TDDQH
VLTMG GSGTS GTLRF HADNG ESFTA TFGVH NYKRW CDIVT
NLAAD ETGMV INQQY YSQKN REEAR ERQLS NYQVK NAKGR
NFQIV YTEAE GNDLH ANLII G
Recombinant lectin protein having Amino acid
sequence of SEQ ID NO: 2
VYKIT VRVYQ TNPDA FFHPV EKTVW KYANG GTWSI TDDQH
VLTMG GSGTS GTLRF HADNG ESFTA TFGVH NYKRW CDIVT
NLAAD ETGMV INQQY YSQKN REEAR ERQLS NYQVK NAKGR
NFQIV YTEAE GNDLH ANLII G
Recombinant lectin protein having Amino acid
sequence of SEQ ID NO: 3
VYKIT VRVYQ TNPDA FFHPV EKTVW KYADG GTWSI
TDDQHVLTMG GSGTS GTLRF HADNG ESFTA TFGVH DYKRW
CDIVT DLAAD ETGMV INQEY YSEKD REEAR ERQNS NYEVK
DAKGR NFEIV YTEAE GNDLH ADLII G

### DETAILED DESCRIPTION OF THE INVENTION

The present invention relates in general to a therapeutically effective combination comprising a recombinant lectin protein and one or more other therapeutic agents.

In certain embodiments there is provided a therapeutically effective combination comprising are combinant lectin protein and one or more other therapeutic agents, wherein the combination is synergistic. In some embodiments, the concentration of the recombinant lectin protein is in the range from 0.5 μg/mL to 100 μg/mL.

### Therapy

The therapeutically effective combination of the present invention is for the prevention of growth or metastasis of tumor, treatment or to cure cancer. According to some embodiments the synergistic combination may be used for prevention or treatment of adenocarcinoma, Squamous cell carcinoma, Transitional cell carcinoma, Basal cell carcinoma, Sarcomas, Lymphomas, epithelial cell carcinomas or non-epithelial cell carcinomas. According to a particular embodiment the combination therapy of the present invention may be used for prevention or treatment of pancreatic cancer, abdominal cancer, liver cancer, prostate cancer, oral cancer, colon cancer, ovary cancer, bladder cancer, kidney cancer, stomach cancer, breast cancer, bone marrow cancer, melanoma, leukemia or central nervous system cancer in a subject.

According to a specific embodiment, the combination therapy of the present invention may be used for prevention or treatment of pancreatic cancer, oral cancer, colon cancer, ovary cancer, bladder cancer, breast cancer.

### Lectin Protein

The recombinant lectin protein is the protein having the amino acid sequence of SEQ ID NO: 1 or, in some embodiments, a protein comprising an amino acid sequence having at least 60% homology to SEQ ID NO: 1.In some embodiments, the recombinant lectin protein may comprise an amino acid sequence having at least 60% homology to SEQ ID NO: 2 or 3. In some embodiments the recombinant lectin protein comprises an amino acid sequence having at least 70%, 80%, 90%, 95%, 96%, 97%, 98%or 99% homology to SEQ ID NO: 1.In some embodiments, the recombinant lectin protein comprises the amino acid sequence of SEQ ID NO: 1, 2 or 3. In some embodiments, the lectin protein comprises fewer than 200 amino acid residues, preferably fewer than 150 amino acid residues.

### Other Therapeutic Agent

In some embodiments, the other therapeutic agent is an anti-cancer agent used in chemotherapy for the treatment of cancer. In some embodiments the other therapeutic agent is selected from alkylating agents such as, e.g., mitomycin C, cyclophosphamide, busulfan, ifosfamide, isosfamide, melphalan, hexamethylmelamine, thiotepa, chlorambucil, mechlorethamine or dacarbazine; antimetabolites such as, e.g., gemcitabine, capecitabine, 5-fluorouracil, cytarabine, 2-fluorodeoxy cytidine, methotrexate, idatrexate, tomudex or trimetrexate; topoisomerase II inhibitors such as, e.g., doxorubicin, epirubicin, etoposide, teniposide or mitoxantrone; topoisomerase I inhibitors such as, e.g., irinotecan (CPT-11), 7-ethyl-10-hydroxy-camptothecin (SN-38) or topotecan; antimitotic drugs such as, e.g., paclitaxel, docetaxel, vinblastine, vincristine or vinorelbine; and platinum derivatives such as, e.g., cisplatin, oxaliplatin, spiroplatinum or carboplatinum.

As per American cancer society (www.cancer.org) a chemotherapeutic agent used for treatment of breast cancers is selected from Paclitaxel Taxol, Docetaxel Taxotere, Doxorubicin, Epirubicin, Cisplatin, Carboplatin, Vinorelbine, Capecitabine, Gemcitabine, Ixabepilone and Eribulin. Likewise, for the treatment of Pancreatic cancers, a chemotherapeutic agent is selected from Gemcitabine, 5-fluorouracil, Oxaliplatin, paclitaxel, Capecitabine, Cisplatin and Irinotecan.Also, for the treatment of cancer of the oral cavity and oropharynx, a chemotherapeutic agent is selected from Cisplatin, Carboplatin, 5-Fluorouracil (5-FU), Paclitaxel, Docetaxel and Hydroxyurea. Similarly for treatment of cancers of the colon or rectum, a chemotherapeutic agent is selected from 5-FU, Capecitabine, Irinotecan, Oxaliplatin and a combination of Trifluridine and Tipiracil. For treatment of cancers of the ovary the drug is selected from Platinum compounds such as Cisplatin or Carboplatin and taxane, such as Paclitaxel or Docetaxel or in combination and for treatment of bladder cancer the chemotherapeutic agent is selected from Gemcitabine, Cisplatin, Methotrexate, Vinblastine, Doxorubicin, Carboplatin or their combinations.

According to the specific embodiment of the present invention, the other therapeutic agent may be selected from Paclitaxel, Cisplatin and Carboplatin for breast cancer or ovary cancer, Cisplatin and 5-FU for oral cancer, Cisplatin and Gemcitabine for bladder cancer, 5-FU and Gemcitabine for pancreatic cancer and 5-FU and Irinotecan for colon cancer.

### Regimen

The therapeutically effective combination may include administration of recombinant lectin protein and the other therapeutic agent to the subject in the same or separate pharmaceutical formulations, and at the same time or at different times wherein the recombinant lectin protein and the other therapeutic agent may be administered simultaneously, separately or sequentially with respect to each other.

The term “simultaneous administration,” as used herein, means that the composition comprising recombinant lectin protein and the other therapeutic agent are administered with a time separation of no more than about 15 minute(s), more specifically no more than about any of 10, 5, or 1 minutes. When recombinant lectin protein and the therapeutic agent are administered simultaneously, the recombinant lectin protein and the therapeutic agent may be contained in the same composition (e.g., a composition comprising both, recombinant lectin protein and the one or more therapeutic agent) or in separate compositions (e.g., the recombinant lectin protein is contained in one composition and the one or more therapeutic agents are contained in another composition).

In the “sequential administration” the other therapeutic agent is administered either prior to or after the administration of the recombinant lectin protein. In the sequential administration the recombinant lectin protein and the other therapeutic agent/s may be administered with a time separation of more than about 15 minutes, more specifically, more than about any of 20, 30, 40, 50 and 60 or more minutes. Either the recombinant lectin protein or the other therapeutic agent/s may be administered first. The recombinant lectin protein and the other therapeutic agent/s are contained in separate compositions, which may be contained in the same or different packages.

In the “separate administration” the recombinant lectin protein and the other therapeutic agent/s may be administered in a sequential manner wherein one is administered first and the other second or vice versa. The time separation between the two administrations is more than 60 minutes.

In another embodiment, there is provided use of recombinant lectin protein for the treatment or prevention of cancer comprising administering recombinant lectin protein having amino acid sequence of SEQ ID NO: 1 in combination with another therapeutic agent, wherein the other therapeutic agent is administered simultaneously, separately or sequentially.

In a specific embodiment, there is the use of recombinant lectin protein for prevention or treatment of cancer in a subject comprising administering to the subject an effective amount of recombinant lectin in combination with other anti-cancer agent selected from an antimetabolite, an alkylating antineoplastic agent, an anti-microtubule agent and a topoisomerase I inhibitor, wherein the antimetabolite is selected from 5-FU, Gemcitabine, Methotrexate, Pemetrexed or Capecitabine, the alkylating antineoplastic agent is a platinum based anti-neoplastic agent selected from Cisplatin or Carboplatin, the anti-microtubule agent is Paclitaxel, Docetaxel, Abraxane or Taxotere and topoisomerase I inhibitor is Irinotecan or Topotecan, and wherein the other anti-cancer agent is administered simultaneously, separately or sequentially.

In yet another specific embodiment there is provide the use of recombinant lectin protein for the treatment or prevention of cancer comprising administering recombinant lectin protein in combination with another anti-cancer agent selected from Paclitaxel, 5-Fluorouracil, Cisplatin, Carboplatin, Irinotecan and Gemcitabine and wherein the other anti-cancer agent is administered simultaneously, separately or sequentially.

Another aspect of the present invention is a method of treatment or prevention of cancer in a subject comprising administering to the subject an effective amount of recombinant protein in combination with another therapeutic agent, wherein the other therapeutic agent is administered simultaneously, separately or sequentially.

In a specific embodiment, there is provided a method of treatment or prevention of cancer in a subject comprising administering to the subject an effective amount of recombinant lectin protein in combination with other anti-cancer agent selected from an antimetabolite, an alkylating antineoplastic agent, an anti-microtubule agent and a topoisomerase I inhibitor, wherein the antimetabolite is selected from 5-FU, Gemcitabine, Methotrexate, Pemetrexed or Capecitabine, the alkylating antineoplastic agent is a platinum based anti-neoplastic agent selected from Cisplatin or Carboplatin, the anti-microtubule agent is Paclitaxel, Docetaxel, Abraxane or Taxotere and the topoisomerase I inhibitor is Irinotecan or Topotecan, and wherein the other anti-cancer agent is administered simultaneously, separately or sequentially.

In yet another specific embodiment, there is provided a method of treatment or prevention of cancer in a subject comprising administering to the subject an effective amount of recombinant lectin protein in combination with another anti-cancer agent selected from Paclitaxel, 5-FU, Cisplatin, Carboplatin, Irinotecan and Gemcitabine and wherein the other anti-cancer agent is administered simultaneously, separately or sequentially

### Exemplary Combinations

The recombinant lectin protein when used standalone exhibited effective cytotoxicity. However when combined with other therapeutic agent, the effect on the test cell lines was highly surprising. The combination not only exhibited additive efficacious effect, but high synergism was also observed. The combinations were highly efficacious for inhibition of growth of cancer cells.

In another specific embodiment of the present invention, there is provide a synergistic combination comprising recombinant lectin protein and another anti-cancer agent selected from Paclitaxel, 5-FU, Carboplatin, Cisplatin, Irinotecan and Gemcitabine. The combination is used for the treatment or prevention of cancer.

The main aspect of the present invention is the synergistic combination comprising recombinant lectin protein having amino acid sequence of SEQ ID NO: 1 and Cisplatin, wherein the combination is used for the treatment or prevention of breast, oral, ovarian or bladder cancer,

Another main aspect of the present invention is the synergistic combination comprising recombinant lectin protein and 5-FU, wherein the combination is used for treatment or prevention of pancreatic, oral or colon cancer.

Additional main aspect of present invention is the synergistic combination comprising recombinant lectin protein and Irinotecan, wherein the combination is used for treatment or prevention of colon cancer.

Yet another main aspect of the present invention is the synergistic combination comprising recombinant lectin protein and Paclitaxel, wherein the combination is used for treatment or prevention of ovarian and breast cancer.

Yet another main aspect of the present invention is the synergistic combination comprising recombinant lectin protein and Gemcitabine, wherein the combination is used for treatment or prevention of bladder and pancreatic cancer.

Another main aspect of the invention is the synergistic combination comprising recombinant lectin protein and carboplatin, wherein the combination is used for treatment or prevention of breast cancer.

According to yet another embodiment the combination therapy may include administration of constituents of the present invention simultaneously with radiation therapy.

### Pharmaceutical Composition

Another aspect of the present invention is a composition comprising recombinant lectin protein in combination with another therapeutic agent and one or more pharmaceutically acceptable excipients.

According to the aspect of the invention the combined preparation is in a suitable pharmaceutically acceptable form/pharmaceutical composition. The preparation can be a suitable oral form or a parenterally administrable form or an implant. The preparation, for example, can be in the form of tablets, capsules, lozenges, suspensions, solutions, emulsions, powders, or syrups for intravenous, intramuscular, intraperitoneal, subcutaneous, intradermal, depot injection, intrathecal, transdermal, sublingual, intrahepatic, oral or by inhalation administration.

According to the aspect of the invention the pharmaceutical composition also comprises one or more pharmaceutically acceptable excipients. Pharmaceutically acceptable excipient refers to non-active pharmaceutical ingredients in the formulation known to the skilled person, such as a stabilizer, a solubilizer, a preservative, disintegrators, binders, fillers, and lubricants or any other known to the person skilled in the art.

The composition used in present invention comprise 50 mM Tris Base (Tromethamine), 150 mM Sodium Chloride with pH 8.0±0.2. However such parenteral composition comprise excipients not limited to Dextrose, Glycerol, Sodium chloride or Mannitol as tonicity modifiers; Ascorbic acid, Acetylcysteine, Sulfurous acid salts (bisulfite, metabisulfite) or Monothioglyercol as antioxidants; Phenol, Metacresol, Benzyl alcohol, Parabens (methyl, propyl, butyl), Chlorobutanol, Thimerosal and Phenylmercuric salts (acetate, borate, nitrate) as antimicrobial agents; Calcium disodium, Ethylenediaminetetra acetic acid (EDTA), Disodium EDTA, Sodium EDTA, Calcium or Diethylenetriaminepenta acetic acid as Chelating agents; Polyoxyethylenesorbitanmonooleate (Tween 80), Sorbitanmonooleate Polyoxyethylenesorbitanmonolaurate (Tween 20), Lecithin, Polyoxyethylenepolyoxypropylene copolymers (Pluronics) or Sorbitantrioleate (span 85) as surfactants; and Propylene glycol, Glycerin, Ethanol, Polyethylene glycol, Sorbitol, Dimethylacetamide or Cremophor EL as co-solvent. Other pharmaceutical compositions are prepared according to the prior knowledge of the person skilled in the art using excipients listed in standard books and acceptable by the regulatory authorities across countries.

The preferable quantity of recombinant lectin protein and the other therapeutic agent in a pharmaceutical composition varies according to the form of composition, route of administration, type, age and genetic make-up of the subject and stage of cancer and the method of treatment.

### Concentrations and Dosages

According to some embodiments, the effective concentration of recombinant lectin protein is in the range from 0.5 μg/mL to 100 μg/mL. In a particular embodiment the effective concentration is in the range from 1 μg/mL to 90 μg/mL. According to some embodiments, the effective concentration of the other therapeutic agent is in the range from 0.001 nM to 1000 μM. A skilled person will be aware of the fact that, in the case of combinations, the effective concentration is the concentration of all the ingredients which, when combined, give a synergistic effect. Thus the effective concentration of recombinant lectin protein will differ based on the other therapeutic agent, method of treatment and cancer type. Similarly, the effective concentration of each therapeutic agent will be different for different cancers, when used along with the recombinant lectin protein or individually. For example, in the present invention, the effective synergistic concentration in the case of ovary cancer cell lines, of recombinant lectin protein was from 1 μg/mL to 20 μg/mL whereas no synergistic effect was seen for breast cancer cell line at a concentration of 2.5 μg/mL of recombinant lectin protein even if Paclitaxel was used as other therapeutic agent in both the case. Similarly, the effective synergistic concentrations of Paclitaxel in both these cancers is different. Thus the concentrations of all active agents vary based on the cancer type and the person skilled in the art is well aware of it.

The present inventors performed several studies on different cancer cell lines to examine the effect of recombinant lectin protein standalone and in combination with therapeutic agent listed above for the treatment of several cancers. The present inventors selected Gemcitabine and 5-FU for Pancreatic cancer, Paclitaxel and carboplatin for Breast cancer, Cisplatin and 5-FU for oral cancer, 5-FU and Irinotecan for colon cancer, Cisplatin and Paclitaxel for ovarian cancer and Cisplatin and Gemcitabine for bladder cancer.

The treatment or prevention may comprise administering a therapeutically effective amount of the lectin to the subject. In some embodiments, the lectin is administered at a dose of from 0.1 to 1000 mg/kg, from 0.5 to 100 mg/kg or from 1 to 50 mg/kg. It will be within the capabilities of the skilled person to determine an amount of lectin to be administered according to the nature of the condition being treated and the subject.

The treatment or prevention may comprise administering a therapeutically effective amount of the other therapeutic agent to the subject. In some embodiments, the other therapeutic agent is administered at a dose of from 1-10,000 mg per unit body surface area (mg/m²), preferably 10-1,000 mg/m², preferably 50-600 mg/m².

The present invention thus also provides a pharmaceutical composition comprising a lectin protein and a pharmaceutically acceptable diluent or excipient. Exemplary diluents and excipients include sterilised water, physiological saline, and/or pharmaceutically acceptable buffer. Administration of the lectin or composition may be by any suitable route, including but not limited to, injection (including intravenous (bolus or infusion), intra-arterial, intraperitoneal, subcutaneous (bolus or infusion), intraventricular, intramuscular, or subarachnoidal), oral ingestion (e.g. of a tablet, gel, lozenge or liquid), inhalation, topical, via a mucosa (such as the oral, nasal or rectal mucosa), by delivery in the form of a spray, tablet, transdermal patch, subcutaneous implant or in the form of a suppository. In some embodiments, a lectin (such as a lectin having the amino acid sequence of SEQ ID NO: 1, 2, 3) or a pharmaceutical composition as described herein is administered to the subject enterally, parenterally or topically. The lectin or pharmaceutical composition may be administered as a dosage form which is solid (such as tablet or capsule), a lyophilized powder, a liquid (such as solution or suspension), a semi-solid or any other form as known to the person skilled in the art. The lectin or the pharmaceutical composition may be administered to the subject by injecting a solution or suspension intravenously, intramuscularly, intraperitoneally, subcutaneously, or intradermally, by depot injection, or it may be administered intrathecally, transdermally, sublingually or by oral, topical or inhalation methods. The subject may be a mammalian subject. In some embodiments, the subject is human. In particular, the subject may be a human subject suffering from cancer.

### EXAMPLES

The examples in the present invention are for demonstration of the effect of SEQ ID NO: 1 standalone and in combination with representative anti-cancer agents against selected cancer cells. The anti-cancer agents selected here represent each class of chemotherapeutic agent that is antimetabolites, alkylating antineoplastic agents, anti-microtubule agents and topoisomerase I inhibitors. Thus the person skilled in the art will be well aware of the fact that any other anti-cancer agent, not included in the examples below but belonging to the class of chemotherapeutic agent listed above, would demonstrate similar synergistic effect with some degree of concentration variations. Therefore, the examples listed below are for demonstration purpose only and do not limit the scope of this invention in any manner.

The cell lines used in the experiments were procured from American Type Culture Collection (ATCC).

### Example 1: Cytotoxic Effect of SEQ ID NO: 1, Gemcitabine and 5-Fluorouracil (5-FU) Standalone, and Synergistic Cytotoxic Effect of SEQ ID NO: 1 in Combination With Gemcitabine and 5-FU on Pancreatic Cancer Cells (PANC-1 Cell Line)

A study was conducted to determine the synergistic cytotoxic effects of SEQ ID NO: 1 in combination with chemotherapeutic agents, Gemcitabine and 5-Fluorouracil, in pancreatic cancer cell line (PANC-1). The study was conducted in two phases, described in Example 1-1 and Example 1-2.

### Example 1-1—Measurement of IC50 Values of Gemcitabine and 5-FU in the Presence of SEQ ID NO: 1 for Cytotoxicity in PANC-1 Cell Lines

A study was performed to determine the effect of the concentration of the recombinant lectin represented by SEQ ID NO: 1 on the IC50 of Gemcitabine for cytotoxicity in PANC-1 cells. Conversely the effect of the concentration of Gemcitabine on the IC50 of the recombinant lectin represented by SEQ ID NO: 1.

Similarly, another study was performed for 5-FU in combination with the recombinant lectin of SEQ ID NO: 1.

The cell were kept in contact with the recombinant lectin of SEQ ID NO: 1 and the chemotherapy agent for 48 h and the cytotoxic effect (percentage cytotoxicity) was measured. The cytotoxic effect was determined by well-known colorimetric MTT assay (Mossman, T. 1983 Rapid colorimetric assay for cellular growth and survival: application to proliferation and cytotoxicity assays Journal of Immunological Methods, volume 65, pages 55-63) for assessing cell metabolic activity.

Based on percentage cytotoxicity data, IC50 values were determined using the software GraphPad Prism version 4.01. These determined IC50 values are given in Table 1 and Table 2 below.

TABLE 1
IC50 values of SEQ ID NO: 1 and Gemcitabine alone and
in combination for cytotoxicity inPANC-1 cell lines

| Concentration of | IC₅₀ of SEQ ID |
|---|---|
| Gemcitabine (μM) | NO: 1 (μg/mL) |
| 0 | 25.060 |
| 0.01 | 55.490 |
| 0.1 | 19.450 |
| 1 | 14.130 |
| 5 | 12.230 |
| 25 | 2.044 |
| 50 | 0.917 |

| Conc. of SEQ ID | IC₅₀ of |
|---|---|
| NO: 1 (μg/mL) | Gemcitabine (μM) |
| 0 | 7.407 |
| 2.5 | 15.940 |
| 5 | 10.790 |
| 10 | 3.036 |
| 20 | 0.039 |
| 40 | 0.038 |
| 80 | 10.610 |

TABLE 2
IC50 values of SEQ ID NO: 1 and 5-Flurouracil (5-FU)
alone and in combination against PANC-1 cell lines

| Concentration of | IC₅₀ of SEQ ID |
|---|---|
| 5-FU (μM) | NO: 1 (μg/mL) |
| 0 | 24.630 |
| 0.1 | 686.900 |
| 1 | 22.490 |
| 10 | 6.842 |
| 50 | 1.690 |
| 100 | 1.070 |
| 250 | 0.098 |
| Conc. of SEQ ID | IC₅₀ of |
| NO: 1 (μg/mL) | 5-FU (μM) |
| 0 | 86.99 |
| 2.5 | 296.80 |
| 5 | 1.409 |
| 10 | 1.257 |
| 20 | 0.869 |
| 40 | 8.454 |
| 80 | 0.725 |

Table 1 and 2 relate to the IC50 of SEQ ID NO: 1 alone or in combination with Gemcitabine or 5-FU. It is observed that IC50 towards pancreatic cancer cells was reduced considerable when recombinant lectin protein having amino acid sequence of SEQ ID NO: 1 was used in combination with Gemcitabine or5-Flurouracil(5-FU).

### Example 1-2—Calculation of CI Values for Gemcitabine and 5-FU With SEQ ID NO: 1

In a further study, PANC-1 cells were treated with Gemcitabine (0.01 μM-50 μM) in combination with SEQ ID NO: 1(2.5 μg/mL-80 μg/mL) for 48 h. The cytotoxic effect on each combination was determined by MTT assay. Similarly another set of cells were treated with 5-FU (0.1 μM-250 μM) in combination with SEQ ID NO: 1 (2.5 μg/mL-80 μg/mL).

Using the same method as in Example 1-1, the IC50 value for Gemcitabine at a given concentration of the recombinant lectin of SEQ ID NO: 1 was measured and the IC50 value for the recombinant lectin of SEQ ID NO: 1 at a given concentration of Gemcitabine was determined. The IC50 value for each of Gemcitabine and the recombinant lectin of SEQ ID NO: 1 alone was also measured. The same was performed for 5-FU in place of Gemcitabine.

Combination Index (CI) values were determined for each drug concentration combination. The CI values were calculated based on the IC50 values using the formula as given in below.

$$CI = \frac{{DA}_{( 50 )}}{{ICA}_{( 50 )}} + \frac{{DB}_{( 50 )}}{{ICB}_{( 50 )}} + \frac{{DA}_{( 50 )} \times {DB}_{( 50 )}}{{ICA}_{( 50 )} \times {ICB}_{( 50 )}}$$

Where,

DA₍₅₀₎=Concentration of drug A in combination with B to produce 50% cytotoxicity

ICA₍₅₀₎=Concentration of drug A alone to produce 50% cytotoxicity

DB₍₅₀₎=Concentration of drug B in combination with A to produce 50% cytotoxicity

ICB₍₅₀₎=Concentration of drug B alone to produce 50% cytotoxicity

Wherein drug A is SEQ ID NO: 1 and drug B is other therapeutic agent that was tested.

Synergism, additive or antagonism were determined based on the following criteria:
CI>1—antagonism is indicated
CI=1—additive effected is indicated
CI<1—synergism is indicated

The results of combination index calculations for study on combination of SEQ ID NO: 1 with Gemcitabine and 5-FU against PANC-1cell lines is tabulated below.

TABLE 3
CI values of combination of SEQ ID NO: 1 and Gemcitabine
after 48 hours in PANC-1 cell line

| | | Concentration of Gemcitabine (μM) | | | | | |
|---|---|---|---|---|---|---|---|
| | | 0.01 | 0.1 | 1 | 5 | 25 | 50 |
| Conc. of | 2.5 | 9.131 | 6.897 | 3.532 | 2.231 | 2.231 | 6.819 |
| SEQ ID NO: | 5 | 4.598 | 3.363 | 1.504 | 0.786 | 0.785 | 3.320 |
| 1(μg/mL) | 10 | 3.929 | 2.842 | 1.205 | 0.572 | 0.572 | 2.804 |
| | 20 | 3.690 | 2.656 | 1.098 | 0.496 | 0.496 | 2.620 |
| | 40 | 2.409 | 1.657 | 0.525 | 0.087 | 0.087 | 1.631 |
| | 80 | 2.267 | 1.547 | 0.461 | 0.042 | 0.042 | 1.521 |

According to the aspect of the invention synergism was observed for both the combinations against pancreatic cell line as tabulated above. Pairs of concentrations which showed synergistic effect (i.e. CI<1) are underlined. For the combination of SEQ ID NO: 1 and Gemcitabine, effective concentration towards synergistic cytotoxic effect was observed for SEQ ID NO: 1 concentration:

For 5 μg/mL, 10 μg/mL and 20 μg/mL of SEQ ID NO: 1 with 5 μM and 25 μM of Gemcitabine

For 40 μg/mL and 80 μg/mL of SEQ ID NO: 1 with 1 μM, 5 μLM and 25 μM of Gemcitabine

Antagonism was observed at all concentrations of SEQ ID NO: 1 with 0.01 μM, 0.1 μM and 50 μM of Gemcitabine. At 2.5 μg/mL of SEQ ID NO: 1, antagonism was observed for all concentrations of Gemcitabine. Antagonism as also observed for 5 μg/mL and 10 μg/mL of SEQ ID NO: 1 with 1 μM of Gemcitabine.

Additive effect was observed at 20 μg/mL of SEQ ID NO: 1 with 1 μM of Gemcitabine.

TABLE 4
CI values of combination of SEQ ID NO: 1 and 5-FU against PANC-1 cell lines

| | | Concentration of 5-FU (μM) | | | | | |
|---|---|---|---|---|---|---|---|
| | | 0.1 | 1 | 10 | 50 | 100 | 250 |
| Conc. of | 2.5 | 126.454 | 28.357 | 28.306 | 28.177 | 30.696 | 28.129 |
| SEQ ID NO: | 5 | 7.440 | 0.944 | 0.941 | 0.932 | 1.099 | 0.929 |
| 1(μg/mL) | 10 | 4.637 | 0.298 | 0.296 | 0.291 | 0.402 | 0.288 |
| | 20 | 3.715 | 0.086 | 0.084 | 0.079 | 0.172 | 0.078 |
| | 40 | 3.604 | 0.060 | 0.059 | 0.054 | 0.145 | 0.052 |
| | 80 | 3.429 | 0.020 | 0.018 | 0.014 | 0.102 | 0.012 |

According to the aspect of the invention synergism was observed for both the combinations against pancreatic cell line as tabulated above. Pairs of concentrations which showed synergistic effect are underlined. For the combination of SEQ ID NO: 1 and 5-FU, effective concentration towards synergistic cytotoxic effect was observed for SEQ ID NO: 1 concentration:

For 5 μg/mL of SEQ ID NO: 1 with 1 μM to 50 μM, and 250 μM of 5-FU.

For 10 μg/mL to 80 μg/mL of SEQ ID NO: 1 with 1 μM to250 μM of 5-FU.

Antagonism was observed at lower concentrations of both SEQ ID NO: 1 and 5-FU.

Additive effect was observed at 5 μg/mL of SEQ ID NO: 1 with 100 OA of 5-FU.

The results of Tables 1 to 4 demonstrate that SEQ ID NO: 1 shows synergistic effects in combination with Gemcitabine and 5-Fluorouracil. The combinations were highly efficacious towards inhibiting the growth of cancer cells. The concentration of SEQ ID NO: 1 used was from about 2.5 μg/mL to about 80 μg/mL. The concentration of other therapeutic agent/s i.e. other than SEQ ID NO: 1, according to the present invention is from 1 μM to about 250 μM.

### Example 2: Cytotoxic Effect of SEQ ID NO: 1, Paclitaxel and Carboplatin Standalone, Synergistic Cytotoxic Effect of SEQ ID NO: 1 in Combination With Paclitaxel and Carboplatin on Breast Cancer Cells (MDA-MB-231 Cell Line)

A study was conducted to determine the synergistic cytotoxic effects of SEQ ID NO: 1in combination with chemotherapeutic agents, paclitaxel and carboplatin, in a breast cancer cell line (MDA-MB-231). The study was conducted in two phases, described in Example 2-1 and Example 2-2.

### Example 2-1—Measurement of IC50 Values for MDA-MB-231 Cells of Paclitaxel and Carboplatin in the Presence of SEQ ID NO: 1

A study was performed to determine the effect of the concentration of the recombinant lectin represented by SEQ ID NO: 1 on the IC50 of Paclitaxel for cytotoxicity in MDA-MB-231 cells. Conversely the effect of the concentration of Paclitaxel on the IC50 of the recombinant lectin represented by SEQ ID NO: 1.

Similarly another study was performed for Carboplatin in combination with the recombinant lectin of SEQ ID NO: 1.

This was performed using the same techniques as in Example 1-1. The determined IC50 values are given in Table 5 and Table 6 below.

TABLE 5
IC50 values of SEQ ID NO: 1 and Paclitaxel alone
and in combination against MDA-MB-231 cell lines.

| Concentration of | IC₅₀ of SEQ ID |
|---|---|
| Paclitaxel (nM) | NO: 1 (μg/mL) |
| 0 | 19.800 |
| 0.001 | 25.370 |
| 0.01 | 21.220 |
| 0.1 | 13.700 |
| 1 | 8.720 |
| 5 | 5.775 |
| 10 | 2.248 |
| Conc. of SEQ ID | IC₅₀ of |
| NO: 1 (μg/mL) | Paclitaxel (nM) |
| 0 | 1.638 |
| 2.5 | 3.980 |
| 5 | 9.307 |
| 10 | 0.389 |
| 20 | 0.123 |
| 40 | 3.56 × 10⁻⁵ |
| 80 | 5.75 × 10⁻¹⁰ |

TABLE 6
IC50 values of SEQ ID NO: 1 and Carboplatin alone
and in combination against MPA-MB-231 cell lines.

| Concentration of | IC₅₀ of SEQ ID |
|---|---|
| Carboplatin (μM) | NO: 1 (μg/mL) |
| 0 | 14.310 |
| 10 | 7.188 |
| 50 | 3.584 |
| 100 | 4.290 |
| 200 | 2.073 |
| 500 | 0.523 |
| 1000 | 0.044 |

| Conc. of SEQ ID | IC₅₀ of |
|---|---|
| NO: 1 (μg/mL) | Carboplatin (μM) |
| 0 | 477.100 |
| 2.5 | 226.100 |
| 5 | 3.985 |
| 10 | 4.925 |
| 20 | 8.444 |
| 40 | 1.421 |
| 80 | 5.874 |

Table 5 and 6 relate to IC50 of SEQ ID NO: 1 alone and in combination with Paclitaxel or Carboplatin. It is observed that IC50 towards breast cancer cells was reduced considerably when SEQ ID NO: 1 was used in combination with Carboplatin or Paclitaxel.

### Example 2-2—Calculation of CI Values for Paclitaxel and Carboplatin With SEQ ID NO: 1

In a further study, MDA-MB-231 cells were treated with Paclitaxel (0.001 nM-10 nM) in combination with SEQ ID NO: 1(2.5 μg/mL-80 μg/mL) for 48 h. Similarly another set of cells were treated with Carboplatin (10 μM-1000 μM) in combination with SEQ ID NO: 1 (2.5 μg/mL-80 μg/mL). Combination Index values were determined for each drug combination and resultant antagonistic, additive or synergistic effect was determined, as described in Example 1-2. The results are tabulated below.

TABLE 7
CI values of combination of SEQ ID NO: 1 and
Paclitaxel against MPA-MB-231 cell line.

| | | Concentration of Paclitaxel (nM) | | | | | |
|---|---|---|---|---|---|---|---|
| | | 0.001 | 0.01 | 0.1 | 1 | 5 | 10 |
| Conc. of | 2.5 | 6.824 | 14.244 | 1.823 | 1.453 | 1.281 | 1.281 |
| SEQ ID NO: | 5 | 6.106 | 12.843 | 1.564 | 1.227 | 1.072 | 1.072 |
| 1(μg/mL) | 10 | 4.803 | 10.305 | 1.094 | 0.819 | 0.692 | 0.692 |
| | 20 | 3.940 | 8.625 | 0.782 | 0.549 | 0.440 | 0.440 |
| | 40 | 3.430 | 7.631 | 0.598 | 0.389 | 0.292 | 0.292 |
| | 80 | 2.819 | 6.441 | 0.378 | 0.197 | 0.114 | 0.114 |

According to the aspect of the invention, synergism was observed for both the combinations against the breast cancer cell line as tabulated above. Pairs of concentrations which showed synergistic effect (i.e. CI<1) are underlined. For the combination of SEQ ID NO: 1 and Paclitaxel, effective concentration towards synergistic cytotoxic effect was observed for SEQ ID NO: 1 concentration:

For 10 μg/mL to 80 μg/mL of SEQ ID NO: 1 with 1 nM to 10 nM of Paclitaxel.

For 20 μg/mL to 80 μg/mL of SEQ ID NO: 1 with 0.1 nM of Paclitaxel.

Antagonism was observed at lower concentrations of both SEQ ID NO: 1 (2.5 μg/mL and 5 μg/mL) and Paclitaxel (0.001 nM and 0.01 nM).

Additive effect was observed at 5 μg/mL of SEQ ID NO: 1 with 5 nM and 10 nM of Paclitaxel and 10 μg/mL of SEQ ID NO: 1 with 0.1 nM of Paclitaxel.

TABLE 8
CI values of combination of SEQ ID NO: 1 and
Carboplatin against MPA-MB-231 cell line.

| | | Concentration of Carboplatin (μM) | | | | | |
|---|---|---|---|---|---|---|---|
| | | 10 | 50 | 100 | 200 | 500 | 1000 |
| Conc. of | 2.5 | 2.005 | 1.214 | 0.515 | 0.518 | 0.529 | 0.507 |
| SEQ ID NO: | 5 | 1.501 | 0.843 | 0.261 | 0.263 | 0.273 | 0.254 |
| 1(μg/mL) | 10 | 1.600 | 0.916 | 0.311 | 0.313 | 0.323 | 0.304 |
| | 20 | 1.290 | 0.687 | 0.154 | 0.157 | 0.165 | 0.148 |
| | 40 | 1.073 | 0.528 | 0.045 | 0.047 | 0.055 | 0.040 |
| | 80 | 1.006 | 0.478 | 0.011 | 0.013 | 0.021 | 0.006 |

The synergism was observed for both the combinations against breast cancer cell line as tabulated above. Pairs of concentrations which showed synergistic effect (i.e. CI<1) are underlined. For the combination of SEQ ID NO: 1 and Carboplatin, effective concentration towards synergistic cytotoxic effect was observed for SEQ ID NO: 1 concentration:

For 2.5 μg/mL to 80 μg/mL of SEQ ID NO: 1 with 100 μM to 1000 μM of Carboplatin.

For 5 μg/mL to 80 μg/mL of SEQ ID NO: 1 with 50 μM of Carboplatin.

Antagonism was observed at 2.5 μg/mL to 20 μg/mL of SEQ ID NO: 1 with 10 μM of Carboplatin. Antagonism was also observed at 2.5 μg/mL of SEQ ID NO: 1 with 50 μM of Carboplatin.

Additive effect was observed at 40 μg/mL to 80 μg/mL of SEQ ID NO: 1 with 10 μM of Carboplatin

Results demonstrated that SEQ ID NO: 1 shows synergistic effects in combination with Paclitaxel or Carboplatin.

### Example 3: Cytotoxic Effect of SEQ ID NO: 1, 5-FU and Cisplatin Standalone and Synergistic Cytotoxic Effect of SEQ ID NO: 1 in Combination With 5-FU and Cisplatin on Oral Cancer Cells (KB Cell Line)

A study was conducted to determine the synergistic cytotoxic effects of SEQ ID NO: 1 in combination with chemotherapeutic agents, 5-FU and Cisplatin, in an oral cancer cell line (KB cell line). The study was conducted in two phases, described in Example 3-1 and Example 3-2.

### Example 3-1—Measurement of IC50 Values for KB Cells of 5-FU and Cisplatin in the Presence of SEQ ID NO: 1

A study was performed to determine the effect of the concentration of the recombinant lectin represented by SEQ ID NO: 1 on the IC50 of5-FU for cytotoxicity in KB cells. Conversely the effect of the concentration of 5-FU on the IC50 of the recombinant lectin represented by SEQ ID NO: 1.

Similarly another study was performed for Cisplatin in combination with the recombinant lectin of SEQ ID NO: 1.

This was performed using the same techniques as in Example 1-1. The determined IC50 values are given in Table 9 and Table 10 below.

TABLE 9
IC50 values of SEQ ID NO: 1 and 5-FU alone
and in combination against KB cell lines

| Concentration of | IC₅₀ of SEQ ID |
|---|---|
| 5-FU (μM) | NO: 1 (μg/mL) |
| 0 | 17.3800 |
| 0.01 | 0.5800 |
| 0.1 | 0.9080 |
| 0.25 | 0.5010 |
| 0.5 | 0.1141 |
| 1 | 0.0555 |
| 5 | 0.0871 |
| Conc. of SEQ ID | IC₅₀ of |
| NO: 1 (μg/mL) | 5-FU (μM) |
| 0 | 0.01498 |
| 5 | 0.00010 |
| 10 | 5.31 × 10⁻⁶ |
| 20 | 1.34 × 10⁻⁵ |
| 30 | 1.19 × 10⁻⁶ |
| 40 | 5.22 × 10⁻¹¹ |
| 90 | 1.40 × 10⁻⁸ |

TABLE 10
IC50 values of SEQ ID NO: 1 and Cisplatin alone
and in combination against KB cell lines

| Concentration of | IC₅₀ of SEQ ID |
|---|---|
| Cisplatin (μM) | NO: 1 (μg/mL) |
| 0 | 38.110 |
| 0.1 | 21.190 |
| 0.25 | 11.240 |
| 0.5 | 2.694 |
| 0.75 | 0.597 |
| 1.0 | 0.316 |
| 1.5 | 0.303 |
| Conc. of SEQ ID | IC₅₀ of |
| NO: 1 (μg/mL) | Cisplatin (μM) |
| 0 | 0.72200 |
| 5 | 0.37490 |
| 10 | 0.26880 |
| 20 | 0.13390 |
| 30 | 0.06616 |
| 40 | 0.00712 |
| 90 | 0.00993 |

Table 9 and 10 relate to IC50 of SEQ ID NO: 1 alone and in combination with 5-FU or Cisplatin. It is observed that IC50 towards oral cancer cells was reduced considerably when SEQ ID NO: 1 was used in combination with 5-FU and Cisplatin.

### Example 3-2—Calculation of CI Values for 5-FU and Cisplatin With SEQ ID NO: 1

In a further study, KB cells which were treated were treated with 5-FU (0.01 μM to 5.0 μM) in combination with SEQ ID NO: 1(5.0 μg/mL-90 μg/mL) for 48 h. Similarly another set of cells were treated with Cisplatin(0.1 μM to 1.5 μM)in combination with SEQ ID NO: 1(5.0 μg/mL-90 μg/mL). Combination Index values were determined for each drug combination and result antagonistic, additive or synergistic effect was determined, as described in Example 1-2.

The results are tabulated below.

TABLE 11
CI values of combination of SEQ ID NO: 1 and 5-FU against KB cell line

| | | Concentration of 5-FU (μM) | | | | | |
|---|---|---|---|---|---|---|---|
| | | 0.01 | 0.1 | 0.25 | 0.5 | 1 | 5 |
| Conc. of | 5 | 0.0406 | 0.0338 | 0.0343 | 0.0335 | 0.0334 | 0.0334 |
| SEQ ID NO: | 10 | 0.0596 | 0.0526 | 0.0532 | 0.0523 | 0.0522 | 0.0522 |
| 1(μg/mL) | 20 | 0.0360 | 0.0292 | 0.0297 | 0.0289 | 0.0288 | 0.0288 |
| | 30 | 0.0136 | 0.0069 | 0.0075 | 0.0066 | 0.0066 | 0.0066 |
| | 40 | 0.0102 | 0.0035 | 0.0041 | 0.0033 | 0.0032 | 0.0032 |
| | 90 | 0.0120 | 0.0054 | 0.0059 | 0.0051 | 0.0050 | 0.0050 |

TABLE 12
CI values of combination of SEQ ID NO:
1 and Cisplatin against KB cell lines

| | | Concentration of Cisplatin (μM) | | | | | |
|---|---|---|---|---|---|---|---|
| | | 0.1 | 0.25 | 0.5 | 0.75 | 1 | 1.5 |
| Conc. of | 5 | 1.364 | 1.135 | 0.845 | 0.699 | 0.571 | 0.577 |
| SEQ ID NO: | 10 | 0.967 | 0.777 | 0.535 | 0.414 | 0.308 | 0.313 |
| 1(μg/mL) | 20 | 0.627 | 0.469 | 0.269 | 0.169 | 0.081 | 0.085 |
| | 30 | 0.543 | 0.394 | 0.204 | 0.109 | 0.026 | 0.030 |
| | 40 | 0.532 | 0.384 | 0.195 | 0.101 | 0.018 | 0.022 |
| | 90 | 0.531 | 0.383 | 0.195 | 0.100 | 0.018 | 0.022 |

According to the aspect of the invention synergism was observed for both the combinations against KB cell lines as tabulated above. Pairs of concentrations which showed synergistic effect (i.e. CI<1) are underlined. The combination of SEQ ID NO: 1 and 5-FU exhibited synergism at all the concentrations (SEQ ID NO: 1—from 5.0 μg/mL to 90 μg/mL and 5-FU —from 0.01 μM to 5 μM) that were studied. Whereas in case of combination of SEQ ID NO: 1 and Cisplatin synergism was observed at concentrations:

10 μg/mL to 90 μg/mL of SEQ ID NO: 1 and 0.1 μM to 1.5 μM of Cisplatin, and

5 μg/mL of SEQ ID NO: 1 and 0.5 μM to 1.5 μM of Cisplatin.

Antagonism was observed at lower concentration of both SEQ ID NO: 1 and Cisplatin.

### Example 4: Cytotoxic Effect of SEQ ID NO: 1, 5-FU and Irinotecan Standalone and Synergistic Cytotoxic Effect of SEQ ID NO: 1 in Combination With 5-FU and Irinotecan on Colon Cancer Cells (HT-29 Cell Line)

A study was conducted to determine the synergistic cytotoxic effects of SEQ ID NO: 1 in combination with chemotherapeutic agents, 5-FU and Irinotecan, in a colon cancer cell line (HT-29). The study was conducted in two phases, described in Example 4-1 and Example 4-2.

### Example 4-1—Measurement of IC50 Values for HT-29 Cells of 5-FU and Irinotecan in Presence of SEQ ID NO: 1

A study was performed to determine the effect of the concentration of the recombinant lectin represented by SEQ ID NO: 1 on the IC50 of 5-FU for cytotoxicity in HT-29 cells. Conversely the effect of the concentration of 5-FU on the IC50 of the recombinant lectin represented by SEQ ID NO: 1.

Similarly another study was performed for Irinotecan in combination with the recombinant lectin of SEQ ID NO: 1.

This was performed using the same techniques as in Example 1-1. The determined IC50 values are given in Table 13 and Table 14 below.

TABLE 13
IC50 of SEQ ID NO: 1 and 5-FU alone and
in combination against HT-29 cell lines

| Concentration of | IC₅₀ of SEQ ID |
|---|---|
| 5-FU (μM) | NO: 1 (μg/mL) |
| 0 | 25.470 |
| 5 | 22.990 |
| 10 | 6.374 |
| 50 | 1.667 |
| 100 | 1.387 |
| 150 | 1.216 |
| 200 | 1.334 |
| Conc. of SEQ ID | IC₅₀ of |
| NO: 1 (μg/mL) | 5-FU (μM) |
| 0 | 137.300 |
| 1 | 172.600 |
| 5 | 25.860 |
| 10 | 4.513 |
| 20 | 0.282 |
| 40 | 1.241 |
| 80 | 4.884 |

TABLE 14
IC50 of SEQ ID NO: 1 and Irinotecan alone
and in combination against HT-29 cell lines

| Concentration of | IC₅₀ of SEQ ID |
|---|---|
| Irinotecan (μM) | NO: 1 (μg/mL) |
| 0 | 18.320 |
| 0.1 | 73.480 |
| 1 | 36.280 |
| 5 | 23.520 |
| 10 | 9.995 |
| 25 | 8.258 |
| 50 | 1.395 |
| Conc. of SEQ ID | IC₅₀ of |
| NO: 1 (μg/mL) | Irinotecan (μM) |
| 0 | 16.200 |
| 1 | 49.240 |
| 5 | 2.779 |
| 10 | 5.449 |
| 20 | 1.338 |
| 40 | 0.278 |
| 80 | 0.167 |

Table 13 and 14 relate to IC50 of SEQ ID NO: 1 alone and in combination with 5-FU or Irinotecan. It was observed that the IC50 towards colon cancer cells was reduced considerably when SEQ ID NO: 1 was used in combination with 5-FU and Irinotecan.

### Example 4-2—Calculation of CI Values for 5-FU and Irinotecan With SEQ ID NO: 1

In a further study, HT-29 cells were treated with 5-FU (5 μM-200 μM) in combination with SEQ ID NO: 1 (1 μg/mL-80 μg/mL) for 48 h. Similarly another set of cells were treated with Irinotecan (0.1 μM-50 μM) in combination with SEQ ID NO: 1 (1 μg/mL-80 μg/mL). Combination Index values were determined for each drug combination and resultant antagonistic, additive, or synergistic effect was determined, as described in Example 1-2. The results are tabulated below.

TABLE 15
CI values of combination of SEQ ID NO:
1 and 5-FU against HT-29 cell lines

| | | Concentration of 5-FU (μM) | | | | | |
|---|---|---|---|---|---|---|---|
| | | 5 | 10 | 50 | 100 | 150 | 200 |
| Conc. of | 1 | 3.294 | 1.261 | 0.965 | 0.907 | 0.920 | 0.970 |
| SEQ ID NO: | 5 | 1.822 | 0.486 | 0.291 | 0.253 | 0.262 | 0.295 |
| 1(μg/mL) | 10 | 1.405 | 0.266 | 0.100 | 0.068 | 0.075 | 0.103 |
| | 20 | 1.380 | 0.253 | 0.089 | 0.057 | 0.064 | 0.092 |
| | 40 | 1.365 | 0.245 | 0.082 | 0.050 | 0.057 | 0.085 |
| | 80 | 1.375 | 0.251 | 0.087 | 0.055 | 0.0619 | 0.0898 |

According to the aspect of the invention, synergism was observed for both the combination against the colon cancer cell line as tabulated above. Pairs of concentrations which showed synergistic effect (i.e. CI<1) are underlined. For the combination of SEQ ID NO: 1 and 5-FU, effective concentration towards synergistic cytotoxic effect was observed for SEQ ID NO: 1 concentration:

For 5 μg/mL to 80 μg/mL of SEQ ID NO: 1 with 10 μM to 200 μM of 5-FU concentration.

For 1 μg/mL of SEQ ID NO: 1 with 50 μM to 200 μM of 5-FU concentration.

Antagonism was observed at all concentrations of SEQ ID NO: 1 with 5 μM 5-FU concentration and at combination comprising 1 μg/mL of SEQ ID NO: 1 and 10 μM 5-FU concentration.

TABLE 16
CI values of combination of SEQ ID NO: 1
and Irinotecan against HT-29 cell lines

| | | Concentration of Irinotecan (μM) | | | | | |
|---|---|---|---|---|---|---|---|
| | | 0.1 | 1 | 5 | 10 | 25 | 50 |
| Conc. of | 1 | 19.321 | 4.894 | 5.723 | 4.446 | 4.117 | 4.082 |
| SEQ ID NO: | 5 | 11.039 | 2.492 | 2.983 | 2.227 | 2.031 | 2.011 |
| 1(μg/mL) | 10 | 8.226 | 1.676 | 2.052 | 1.472 | 1.323 | 1.307 |
| | 20 | 5.243 | 0.811 | 1.065 | 0.673 | 0.572 | 0.562 |
| | 40 | 4.860 | 0.700 | 0.939 | 0.571 | 0.476 | 0.466 |
| | 80 | 3.347 | 0.261 | 0.438 | 0.165 | 0.095 | 0.087 |

The synergistic effect was observed for both the combinations against the colon cancer line as tabulated above. Pairs of concentrations which showed synergistic effect (i.e. CI<1) are underlined. For the combination of SEQ ID NO: 1 and Irinotecan, effective concentration towards synergistic cytotoxic effect was overserved for SEQ ID NO: 1 concentration:

For 20 μg/mL to 80 μg/mL of SEQ ID NO: 1 with 1 μM-50 μM (except 5 μM) of Irinotecan concentration.

Antagonism was observed with SEQ ID NO: 1 concentration at 1 μg/mL to 80 μg/mL with 0.1 μM or Irinotecan and at 1 μg/mL-10 μg/mL of SEQ ID NO: 1 with 0.01 to 50 μM of Irinotecan

Additive effect was observed at 20 μg/mL of SEQ ID NO: 1 with 5 μM of Irinotecan.

### Example 5: Cytotoxic Effect of SEQ ID NO: 1, Cisplatin and Paclitaxel Standalone and Synergistic Cytotoxic Effect of SEQ ID NO: 1 in Combination With Cisplatin and Paclitaxel on Ovarian Cancer Cells (PA-1 Cell Line)

A study was conducted to determine the synergistic cytotoxic effects of SEQ ID NO: 1 in combination with chemotherapeutic agents, Cisplatin and Paclitaxel, in ovarian cancer cell line (PA-1 cell line). The study was conducted in two phases, described in Example 5-1 and Example 5-2.

### Example 5-1—Measurement of IC50 Values of Cisplatin and Paclitaxel in the Presence of SEQ ID NO: 1 for Cytotoxicity in PA-1 Cell Lines

A study was performed to determine the effect of the concentration of the recombinant lectin represented by SEQ ID NO: 1 on the IC50 of Cisplatin for cytotoxicity in MDA-MB-231 cells. Conversely the effect of the concentration of Cisplatin on the IC50 of the recombinant lectin represented by SEQ ID NO: 1.

Similarly another study was performed for Paclitaxel in combination with the recombinant lectin of SEQ ID NO: 1.

This was performed using the same techniques as in Example 1-1. The determined IC50 values are given in Table 17 and Table 18 below.

TABLE 17
IC50 of SEQ ID NO: 1 and Cisplatin alone and
in combination against PA-1 cell lines

| Concentration of | IC₅₀ of SEQ ID |
|---|---|
| Cisplatin (μM) | NO: 1 (μg/mL) |
| 0 | 12.070 |
| 0.01 | 8.520 |
| 0.1 | 6.033 |
| 0.25 | 4.455 |
| 0.5 | 0.912 |
| 1 | 0.321 |
| 5 | 0.0036 |
| Conc. of SEQ ID | IC₅₀ of |
| NO: 1 (μg/mL) | Cisplatin (μM) |
| 0 | 0.08566 |
| 1 | 0.716 |
| 2.5 | 0.314 |
| 5 | 0.093 |
| 10 | 0.0042 |
| 15 | 0.0005 |
| 20 | 0.0001 |

TABLE 18
IC50 of SEQ ID NO: 1 and Paclitaxel alone
and in combination against PA-1 cell lines.

| Concentration of | IC₅₀ of SEQ ID |
|---|---|
| Paclitaxel (nM) | NO: 1 (μg/mL) |
| 0 | 8.822 |
| 0.01 | 5.794 |
| 0.1 | 6.545 |
| 0.25 | 4.893 |
| 0.5 | 3.917 |
| 1 | 3.031 |
| 5 | 0.709 |
| Conc. of SEQ ID | IC₅₀ of |
| NO: 1 (μg/mL) | Paclitaxel (nM) |
| 0 | 4.044 |
| 1 | 3.622 |
| 2.5 | 3.478 |
| 5 | 0.222 |
| 10 | 5.37 × 10⁻⁴ |
| 15 | 4.13 × 10⁻⁶ |
| 20 | 1.02 × 10⁻⁶ |

Table 17 and 18 relate to IC50 values of SEQ ID NO: 1alone and in combination with Cisplatin or Paclitaxel. It is observed that IC50 against ovarian cancer cells was reduced considerably when SEQ ID NO: 1 was used in combination with Cisplatin or Paclitaxel.

### Example 5-2—Calculation of CI Values for Cisplatin and Paclitaxel With SEQ ID NO: 1

In a further study, PA-1 cells were treated with Cisplatin(0.01 μM-5 μM) in combination with SEQ ID NO: 1(1 μg/mL-20 μg/mL) for 48 h. Similarly another set of cells were treated with Paclitaxel (0.01 nM-5 nM) in combination with SEQ ID NO: 1 (1 μg/mL-20 μg/mL). Combination Index values were determined for each drug combination and resultant antagonistic, additive or synergistic effect was determined, as described in Example 1-2. The results are tabulated below.

TABLE 19
CI values of combination of SEQ ID NO:
1 and Cisplatin against PA-1 cell lines

| | | Concentration of Cisplatin (μM) | | | | | |
|---|---|---|---|---|---|---|---|
| | | 0.01 | 0.1 | 0.25 | 0.5 | 1 | 5 |
| Conc. of | 1 | 2.131 | 1.330 | 0.891 | 0.714 | 0.707 | 0.706 |
| SEQ ID NO: | 2.5 | 1.753 | 1.049 | 0.663 | 0.507 | 0.501 | 0.500 |
| 1(μg/mL) | 5 | 1.513 | 0.870 | 0.518 | 0.376 | 0.370 | 0.369 |
| | 10 | 0.974 | 0.469 | 0.192 | 0.081 | 0.076 | 0.076 |
| | 15 | 0.884 | 0.402 | 0.138 | 0.032 | 0.027 | 0.027 |
| | 20 | 0.836 | 0.367 | 0.109 | 0.005 | 0.0009 | 0.0004 |

According to the aspect of the invention, synergism was observed for both the combinations against the ovarian cancer cell line as tabulated above. Pairs of concentrations which showed synergistic effect (i.e. CI<1) are underlined. For the combination of SEQ ID NO: 1 and Cisplatin, effective concentration towards synergistic cytotoxic effect was observed for SEQ ID NO: 1 concentration:

For all concentrations tested (1 μg/mL to 20 μg/mL) of SEQ ID NO: 1 with 0.25 μM to 5 μM of Cisplatin.

For 5 μg/mL to 20 μg/mL of SEQ ID NO: 1 with 0.1 μM of Cisplatin.

For 10 μg/mL to 20 μg/mL of SEQ ID NO: 1 with 0.01 μM of Cisplatin.

Antagonism was observed at 1 μg/mL to 5 μg/mL of SEQ ID NO: 1 with 0.01 μM of Cisplatin.

Antagonism was observed at 1 μg/mL of SEQ ID NO: 1 with 0.1 μM of Cisplatin.

Additive effect was observed at 2.5 μg/mL of SEQ ID NO: 1 with 0.1 μM of Cisplatin.

TABLE 20
CI values of combination of SEQ ID NO: 1
and Paclitaxel against PA-1 cell lines

| | | Concentration of Paclitaxel (nM) | | | | | |
|---|---|---|---|---|---|---|---|
| | | 0.01 | 0.1 | 0.25 | 0.5 | 1 | 5 |
| Conc. of | 1 | 2.141 | 2.082 | 0.748 | 0.657 | 0.657 | 0.657 |
| SEQ ID NO: | 2.5 | 2.302 | 2.240 | 0.838 | 0.742 | 0.742 | 0.742 |
| 1(μg/mL) | 5 | 1.947 | 1.892 | 0.640 | 0.555 | 0.555 | 0.555 |
| | 10 | 1.737 | 1.686 | 0.523 | 0.444 | 0.444 | 0.444 |
| | 15 | 1.547 | 1.499 | 0.417 | 0.344 | 0.344 | 0.344 |
| | 20 | 1.048 | 1.009 | 0.140 | 0.080 | 0.0803 | 0.0803 |

According to the aspect of the invention, synergism was observed for both the combinations against the ovarian cancer cell line as tabulated above. Pairs of concentrations which showed synergistic effect (i.e. CI<1) are underlined. For the combination of SEQ ID NO: 1 and Paclitaxel, effective concentration towards synergistic cytotoxic effect was observed for SEQ ID NO: 1 concentration:

For all concentrations tested (1 μg/mL to 20 μg/mL) of SEQ ID NO: 1 with 0.25 nM to 5 nM of Paclitaxel.

Antagonism was observed at 1 μg/mL to 15 μg/mL of SEQ ID NO: 1 with 0.01 nM to 0.1 nM of Paclitaxel. Additive effect was observed at 20 μM of SEQ ID NO: 1 with 0.01 nM to 0.1 nM of Paclitaxel.

Results demonstrated that SEQ ID NO: 1 shows synergistic effects in combination with Cisplatin or Paclitaxel.

### Example 6: Cytotoxic Effect of SEQ ID NO: 1, Cisplatin and Gemcitabine Standalone and Synergistic Cytotoxic Effect of SEQ ID NO: 1 in Combination With Cisplatin and Gemcitabine on Bladder Cancer Cells (T24 Cell Line)

A study was conducted to determine the synergistic cytotoxic effects of SEQ ID NO: 1 in combination with chemotherapeutic agents, Cisplatin and Gemcitabine, in a bladder cancer cell line (T24 cell line). The study was conducted in two phases, described in Example 6-1 and Example 6-2.

### Example 6-1—Measurement of IC50 Values for T24 Cells of Cisplatin and Gemcitabine in the Presence of SEQ ID NO: 1

A study was performed to determine the effect of the concentration of the recombinant lectin represented by SEQ ID NO: 1 on the IC50 of Cisplatin for cytotoxicity in T24 cells. Conversely the effect of the concentration of Cisplatin on the IC50 of the recombinant lectin represented by SEQ ID NO: 1.

Similarly another study was performed for Gemcitabine in combination with the recombinant lectin of SEQ ID NO: 1.

This was performed using the same techniques as in Example 1-1. The determined IC50 values are given in Table 21 and Table 22 below.

TABLE 21
IC50 of SEQ ID NO: 1 and Cisplatin alone
and in combination against T24 cell lines

| Concentration of | IC₅₀ of SEQ ID |
|---|---|
| Cisplatin (μM) | NO: 1 (μg/mL) |
| 0 | 11.470 |
| 1 | 11.460 |
| 10 | 2.375 |
| 25 | 1.062 |
| 50 | 1.081 |
| 100 | 0.944 |
| 500 | 0.716 |
| Conc. of SEQ ID | IC₅₀ of |
| NO: 1 (μg/mL) | Cisplatin (μM) |
| 0 | 39.020 |
| 2.5 | 22.870 |
| 5 | 3.79500 |
| 10 | 0.09708 |
| 20 | 0.03436 |
| 40 | 0.08363 |
| 80 | 0.01342 |

TABLE 22
IC50 of SEQ ID NO: 1 & Gemcitabine alone
& in combination against T24 cell lines

| Concentration of | IC₅₀ of SEQ ID |
|---|---|
| Gemcitabine (μM) | NO: 1 (μg/mL) |
| 0 | 10.350 |
| 1 | 2.242 |
| 10 | 0.892 |
| 50 | 0.626 |
| 100 | 0.155 |
| 200 | 0.149 |
| 300 | 0.029 |

| Conc. of SEQ ID | IC₅₀ of |
|---|---|
| NO: 1 (μg/mL) | Gemcitabine (μM) |
| 0 | 15.420 |
| 2.5 | 0.802 |
| 5 | 0.14090 |
| 10 | 0.00032 |
| 20 | 0.00002 |
| 40 | 6.05 × 10⁻¹⁹ |
| 80 | 7.18 × 10⁻¹⁰ |

Table 21 and 22 relate to IC50 of SEQ ID NO: 1 alone and in combination with Cisplatin and Gemcitabine. The IC50 against bladder cancer cells was reduced considerably when SEQ ID NO: 1 was used in combination with Cisplatin and Gemcitabine.

### Example 6-2—Calculation of CI Values for Cisplatin and Gemcitabine With SEQ ID NO: 1

In a further study, T24 cells were treated with Cisplatin (1 μM-500 μM) in combination with SEQ ID NO: 1(2.5 μg/mL-80 μg/mL) for 48 h. Similarly another set of cells were treated with Gemcitabine (1 μM-300 μM) in combination with SEQ ID NO: 1 (2.5 μg/mL-80 μg/mL). Combination Index values were determined for each drug combination and resultant antagonistic, additive or synergistic effect was determined, as described in Example 1-2. The results are tabulated below.

TABLE 23
CI values of combination of SEQ ID NO:
1 and Cisplatin against T24 cells lines

| | | Concentration of Cisplatin (μM) | | | | | |
|---|---|---|---|---|---|---|---|
| | | 1 | 10 | 25 | 50 | 100 | 500 |
| Conc. of | 2.5 | 2.171 | 1.194 | 1.004 | 1.001 | 1.003 | 1.000 |
| SEQ ID NO: | 5 | 0.915 | 0.324 | 0.210 | 0.208 | 0.210 | 0.207 |
| 1(μg/mL) | 10 | 0.733 | 0.199 | 0.095 | 0.094 | 0.095 | 0.093 |
| | 20 | 0.736 | 0.201 | 0.097 | 0.095 | 0.097 | 0.095 |
| | 40 | 0.717 | 0.188 | 0.085 | 0.083 | 0.085 | 0.083 |
| | 80 | 0.685 | 0.166 | 0.065 | 0.063 | 0.065 | 0.063 |

According to the aspect of the invention, synergism was observed for both the combinations against the bladder cancer cell line as tabulated above. Pairs of concentrations which showed synergistic effect (i.e. CI<1) are underlined. For the combination of SEQ ID NO: 1 and Cisplatin, effective concentration towards synergistic cytotoxic effect was observed for SEQ ID NO: 1 concentration:

For 5 μg/mL to 80 μg/mL of SEQ ID NO: 1 with all tested concentrations (1 μM-500 μM) of Cisplatin.

Antagonism was observed at 2.5 μM of SEQ ID NO: 1 with 1 μM and 10 μM of Cisplatin.

Additive effect was observed at 2.5 μg/mL of SEQ ID NO: 1 with 25 μM-500 μM of Cisplatin.

TABLE 24
CI values of combination SEQ ID NO: 1
and Gemcitabine against T24 cell lines

| | | Concentration of Gemcitabine (μM) | | | | | |
|---|---|---|---|---|---|---|---|
| | | 1 | 10 | 50 | 100 | 200 | 300 |
| Conc. of | 2.5 | 0.280 | 0.228 | 0.217 | 0.217 | 0.217 | 0.217 |
| SEQ ID NO: | 5 | 0.143 | 0.096 | 0.086 | 0.086 | 0.086 | 0.086 |
| 1(μg/mL) | 10 | 0.116 | 0.070 | 0.061 | 0.060 | 0.060 | 0.060 |
| | 20 | 0.068 | 0.024 | 0.015 | 0.015 | 0.015 | 0.015 |
| | 40 | 0.067 | 0.024 | 0.014 | 0.014 | 0.014 | 0.014 |
| | 80 | 0.055 | 0.012 | 0.003 | 0.003 | 0.003 | 0.003 |

The synergism was observed for both the combinations against bladder cancer cell line as tabulated above. Pairs of concentrations which showed synergistic effect (i.e. CI<1) are underlined. For the combination of SEQ ID NO: 1 and Gemcitabine, synergistic cytotoxic effect was observed at all concentrations (2.5 μM-80 μM of SEQ ID NO: 1 with 1 μM-300 μM of Gemcitabine.

### Summary of Examples

As an overview the concentrations at which synergism was observed in the cell lines tested for present invention is summarized in the table below:

TABLE 25
Concentrations of SEQ ID NO: 1 in combination with different therapeutic agents
showing synergistic effect on oral, colon, ovary and bladder cancer cell lines.

| | Cell | Therapeutic | | | Conc. of other | |
|---|---|---|---|---|---|---|
| Cancer | Lines | agents | Conc. of SEQ ID NO: 1 | | therapeutic agents | |
| Pancreas | PANC-1 | Gemcitabine | 5 to 20 | μg/mL | 5 μM to 25 μM | |
| | | | 40 to 80 | μg/mL | 1 μM to 25 μM | |
| | | 5-FU | 10 to 80 | μg/mL | 1 μM to 250 μM | |
| | | | 5 | μg/mL | 1 to 50 μM &250 μM | |
| Breast | MDA-MB-231 | Paclitaxel | 20 to 80 | μg/mL | 0.1 nM to 10 nM | |
| | | | 10 | μg/mL | 1 nM to 10 nM | |
| | | Carboplatin | 5 to 80 | μg/mL | 50 μM to 1000 μM | |
| | | | 2.5 | μg/mL | 100 μM to 1000 μM | |
| Oral | KB | 5-FU | 5 to 90 | μg/mL | 0.01 to 5 | μM |
| | | Cisplatin | 10 to 90 | μg/mL | 0.1 to 1.5 | μM |
| | | | 5 | μg/mL | 0.5 to 1.5 | μM |
| Colon | HT-29 | 5-FU | 5 to 80 | μg/mL | 10 to 200 | μM |
| | | | 1 | μg/mL | 50 to 200 | μM |
| | | Irinotecan | 40 to 80 | μg/mL | 1 to 50 | μM |
| | | | 20 | μg/mL | 1 and 10 to 50 μM | |
| Ovary | PA-1 | Cisplatin | 1 to 2.5 | μg/mL | 0.25 to 5 | μM |
| | | | 5 | μg/mL | 0.1 to 5 | μM |
| | | | 10 to 20 | μg/mL | 0.01 to 5 | μM |
| | | Paclitaxel | 1 to 20 | μg/mL | 0.25 to 5 | nM |
| Bladder | T-24 | Cisplatin | 5 to 80 | μg/mL | 1 to 500 | μM |
| | | Gemcitabine | 2.5 to 80 | μg/mL | 1 to 300 | μM |

1. A therapeutically effective combination comprising a recombinant lectin protein derived from Sclerotium rolfsii lectin and one or more other therapeutic agent, wherein the combination is synergistic and wherein the concentration of recombinant lectin protein is in the range from 0.5 μg/mL to 100 μg/m L.

2. The therapeutically effective combination according to claim 1, wherein the combination is used for prevention or treatment of cancer in a subject.

3. Use of a recombinant lectin protein for the treatment or prevention of cancer in a subject comprising administration of therapeutically effective amount of the recombinant lectin protein derived from Sclerotium rolfsii lectin to the subject wherein the recombinant lectin protein is administered in combination with one or more other therapeutic agent to the subject, and wherein the other therapeutic agent is administered simultaneously, separately or sequentially.

4. A method for treatment or prevention of cancer in a subject comprising administering to the subject effective amount of recombinant lectin protein derived from Sclerotium rolfsii lectin in combination with one or more other therapeutic agent to the subject, and wherein the other therapeutic agent is administered simultaneously, separately or sequentially.

5. A combination therapy for prevention, treatment or to cure cancer or for proliferation in a subject using recombinant lectin protein, wherein the combination therapy comprises administration of recombinant lectin protein in combination with one or more other therapeutic agent, and wherein the other therapeutic agent is administered simultaneously, separately or sequentially.

6. The therapeutically effective combination according to claim 1, wherein the other therapeutic agent is an anticancer agent selected from an antimetabolite, an alkylating antineoplastic agent, an anti-microtubule agent and/or a topoisomerase I inhibitor.

7. (canceled)

8. The therapeutically effective combination according to claim 6, wherein the antimetabolite is selected from 5-Flurouracil (5-FU), Gemcitabine, Methotrexate, Pemetrexed or Capecitabine; the alkylating neoplastic agent is a platinum based anti-neoplastic agent selected from cisplatin or carboplatin; the anti-microtubule agent is selected from Paclitaxel, Docetaxel, Abraxane or Taxotere; and the topoisomerase I inhibitor is selected from Irinotecan or Topotecan.

9.-11. (canceled)

12. The therapeutically effective combination according to claim 8, wherein the anticancer agent is cisplatin and wherein the combination is used for the treatment or prevention of oral, ovarian or bladder cancer in a subject.

13. The therapeutically effective combination according to claim 8, wherein the anticancer agent is 5-FU and wherein the combination is used for treatment or prevention of oral, pancreatic or colon cancer in a subject.

14. The therapeutically effective combination according to claim 8, wherein the anticancer agent is Irinotecan and wherein the combination is used for treatment or prevention of colon cancer in a subject.

15. The therapeutically effective combination according to claim 8, wherein the anticancer agent is Paclitaxel and wherein the combination is used for treatment or prevention of ovarian or breast cancer in a subject.

16. The therapeutically effective combination according to claim 8, wherein the anticancer agent is Gemcitabine and wherein the combination is used for treatment or prevention of bladder or pancreatic cancer in a subject.

17. The therapeutically effective combination according to claim 6, wherein the anticancer agent is carboplatin and wherein the combination is used for treatment or prevention of breast cancer in a subject.

18. The therapeutically effective combination according to claim 1, wherein the recombinant lectin protein is a protein having amino acid sequence of SEQ ID NO:
1, 2 or 3 or having at least 60%, 70%, 80%, 90%, 95%, 95%, 96%, 97%, 98%, or 99% homology to SEQ ID NO: 1, 2 or 3.

19.-20. (canceled)

21. The therapeutically effective combination according to claim 1, or the combination therapy according to any one of claims 5 or 6 to 20, wherein the concentration of the other therapeutic agent is in the range from 0.001 nM to 1000 μM.

22. The therapeutically effective combination according to claim 12, wherein the concentration of Cisplatin is in the range of 0.01 μM to 500 μM.

23. The therapeutically effective combination according to claim 13, wherein the concentration of 5-Flurouracil (5-FU) is in the range of 0.01 μM to 250 μM.

24. The therapeutically effective combination according to claim 14, wherein the concentration of Irinotecan is in the range of 0.1 μM to 50 μM.

25. The therapeutically effective combination according to claim 15, wherein the concentration of Paclitaxel is in the range of 0.001 nM to 10 nM.

26. The therapeutically effective combination according to claim 16, wherein the concentration of Gemcitabine is in the range of 0.01 μM to 300 μM.

27. The therapeutically effective combination according to claim 17, wherein the concentration of Carboplatin is in the range of 10 μM to 1000 μM.

28. The therapeutically effective combination according to claim 1, wherein the combination, of the recombinant lectin protein and the other therapeutic agent, is a composition.

29. The therapeutically effective combination according to claim 28, wherein the composition further comprises one or more pharmaceutically acceptable excipients.

30. A recombinant lectin protein in combination with one or more other therapeutic agent for the treatment or prevention of cancer wherein the other therapeutic agent is selected from one or more of 5-Flurouracil (5-FU), Gemcitabine, cisplatin, Paclitaxel, carboplatin or Irinotecan.

31. A therapeutic agent for the treatment or prevention of cancer, in combination with a recombinant lectin protein, wherein the therapeutic agent is selected from one or more of 5-Flurouracil (5-FU), Gemcitabine, cisplatin, Paclitaxel, carboplatin or Irinotecan.


SEQUENCE LISTING (3 sequences)

SEQ ID NO: 1 - protein, 141 aa, Artificial Sequence
  Synthetic
TYKITVRVYQTNPDAFFHPVEKTVWKYANGGTWTITDDQHVLTMGGSGTSGTLRFHADNGESFTATFGVHNYKRWCDIVTNLAADETGMVINQQYYSQKNREEARERQLSNYQVKNAKGRNFQIVYTEAEGNDLHANLIIG

SEQ ID NO: 2 - protein, 141 aa, Artificial Sequence
  Synthetic
VYKITVRVYQTNPDAFFHPVEKTVWKYANGGTWSITDDQHVLTMGGSGTSGTLRFHADNGESFTATFGVHNYKRWCDIVTNLAADETGMVINQQYYSQKNREEARERQLSNYQVKNAKGRNFQIVYTEAEGNDLHANLIIG

SEQ ID NO: 3 - protein, 141 aa, Artificial Sequence
  Synthetic
VYKITVRVYQTNPDAFFHPVEKTVWKYADGGTWSITDDQHVLTMGGSGTSGTLRFHADNGESFTATFGVHDYKRWCDIVTDLAADETGMVINQEYYSEKDREEARERQNSNYEVKDAKGRNFEIVYTEAEGNDLHADLIIG